(19) United States
(12) Patent Application Publication
(10) Pub. No.: US 2025/0288682 A1
(43) Pub. Date: Sep. 18, 2025

(54) DEVELOPMENT OF NANO-ENCAPSULATED FATTY-ACYL CONJUGATED COLCHICINE

(71) Applicant: University of Virginia Patent Foundation, Charlottesville, VA (US)

(72) Inventors: Mark Kester, Afton, VA (US); Anuradha Illendula, Crozet, VA (US)

(21) Appl. No.: 18/861,187

(22) PCT Filed: Apr. 26, 2023
(86) PCT No.: PCT/US2023/066269

§ 371 Date: Oct. 28, 2024

Related U.S. Application Data
(60) Provisional application No. 63/335,106, filed on Apr. 26, 2022.

(51) Int. Cl. A61K 47/54; A61K 9/127; A61K 9/1277; A61K 47/68

(52) U.S. Cl. CPC A61K 47/542; A61K 9/127; A61K 9/1277; A61K 47/6849

(57) ABSTRACT

Provided are compositions that include colchicine conjugated to behenic acid, optionally wherein the colchicine conjugated to behenic acid is encapsulated by a nanoliposome. In some embodiments, the colchicine is conjugated to behenic acid at the acetamide position of a B-ring of colchicine, the nanoliposome includes a lipid component comprising DSPC, DOPE, and DSPE-PEG, and cholesterol; and/or the colchicine conjugated to behenic acid is present in the nanoliposome in an amount of at least about 400, 500, 600, or more than 600 pg/mL. Also provided are methods for preparing behenic acid-conjugated colchicine derivatives, methods for preparing lipid nanoparticle-encapsulated colchicine derivatives, and methods for using the same to treat and/or prevent inflammatory and/or cardiovascular diseases, disorders, and/or conditions.

### CROSS REFERENCE TO RELATED APPLICATION

The presently disclosed subject matter claims the benefit of U.S. Provisional Patent Application Ser. No. 63/335,106, filed Apr. 26, 2022, the disclosure of which incorporated herein by reference in its entirety.

### TECHNICAL FIELD

The presently disclosed subject matter relates generally to compositions comprising nano-encapsulated fatty-acyl conjugated hydrophilic drugs, including but not limited to colchicine, prodrugs thereof, and metabolites thereof, and methods for using the same for treating and/or preventing inflammatory and/or cardiovascular diseases, disorders, and/or conditions.

### BACKGROUND

Myocardial infarction (MI) is one of the cardiovascular diseases caused by the rupture of atherosclerotic plaque, which results in the necrosis and apoptosis of cardiomyocytes damaging a large area of myocardium tissue. The rapid and massive loss of myocardial muscle activates an intense inflammatory response, which exerts a deleterious effect at the onset of reperfusion. It contributes to infarct size and the cardiac re-modeling process, leading to heart failure. Therefore, inflammation appears to be a promising therapeutic target in patients with MI. In this context, colchicine is a potent drug in treating MI administered orally. This molecule exerts various anti-mitotic and anti-inflammatory effects, inhibiting neutrophil chemoattraction, the inflammasome network, and proinflammatory cytokines. Colchicine has been successfully evaluated in a large-scale clinical trial for mitigating cardiovascular events, including MI and cardiovascular diseases (CVD). There have been over four clinical trials (COLCOT, COPS, COPE, LoDoCo, and LoCoDo2) evaluating colchicine's efficacy in treating CVD. They concluded that colchicine had efficacy in treating CVD in low doses (0.5-0.6 mg/kg maintenance dose). Still, they had the potential for significant side effects on the kidneys, GI tract, and lungs due to drugMOA and administration requirements (Deftereos et al., 2022).

Colchicine suppresses cytokine secretion, chemokine secretion, and platelet aggregation. Efficacy is limited by toxicity and low bioavailability. Colchicine has a narrow therapeutic index and is subject to P-glycol-protein (p-gp) drug resistance mechanisms. Colchicine is a class III drug (Biopharmaceutical Classification System; Amidon et al., 1995), characterized by high solubility and low permeability (log P of about 1.5; Roubille et al., 2013). The administration/delivery of active pharmaceutical agents exhibiting these limitations is often enhanced by nanotechnology and prodrug approaches. To overcome its inherent limitations, colchicine is altered to a prodrug and loaded into nanocarriers, which can effectively reduce off-target effects and increase cycle times compared to freely administered small molecules. Prodrugs can improve the properties of the parent drug by improving the plasma life and reducing toxicities. For example, Singh et al., 2021 demonstrated that conjugating lisofylline with fatty acids like linoleic acid, oleic acid, palmitic acid, and α-lipoic acid can improve the plasma half-life of lisofylline by six-fold. Thus, developing prodrugs aims to improve chemical properties like solubility and stability.

Additionally, prodrugs can improve PK/PD properties, such as absorption, distribution, selective delivery, and decreased pre-systemic metabolism. The fatty acid-conjugated prodrugs are biocompatible, limiting the surge of undesired toxicity. Naturally available fatty acids are composed of different degrees of unsaturation, chain lengths, and configurations. Fatty acids (FAs) are lipophilic biomolecules and are important phospholipidic membrane components. They facilitate crossing biological barriers, such as the gastrointestinal tract (Porter et al., 2007), and improve bioavailability. These are composed of hydrocarbon chains with a methyl group at one end and a reactive carboxylic acid at the other, readily reacting with primary hydroxyl (—OH) and amine (—NH₂) groups on drug molecules to form labile ester and amide linkages. The release rate of these prodrugs can be altered by varying the chain length of the fatty acids, Koseki et al., 2016.

Several different applications of colchicine nanoparticles have been reported (see e.g., Crielaard et al. 2011; Crielaard et al. 2012; Joshi et al. 2016; AbouAitah et al. 2019; Parashar et al. 2019; Shchegravina et al. 2019; Mohamed et al. 2020; Nasr et al. 2020; Zhang et al. 2020; Elsewedy et al. 2021; and Wang et al. 2022) facilitated by encapsulating colchicine or colchicine prodrug and self-assembled colchicine prodrugs nanocarrier. Among these studies, Wang et al., 2022 used calcium nanoparticles loaded with colchicine to attenuate acute myocardial infarction injury. However, biocompatibility, stability, and long-term cytotoxicity are rising concerns of inorganic nanoparticles. Unlike inorganic materials, polymers and lipids can be readily tailored to be biocompatible, biodegradable, and chemically or mechanically stable.

Disclosed herein are The presently disclosed nanoscale formulations are pH-resistant, first-pass-resistant, and offer the potential for enteral delivery and arterial targeting.

### SUMMARY

This summary lists several embodiments of the presently disclosed subject matter, and in many cases lists variations and permutations of these embodiments of the presently disclosed subject matter. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this summary does not list or suggest all possible combinations of such features.

The presently disclosed subject matter relates in some embodiments to compositions comprising, consisting essentially of, or consisting of colchicine conjugated to behenic acid, stearic acid, palmitic acid and oleic acid. In some embodiments, the colchicine conjugated to behenic acid, stearic acid, palmitic acid and/or oleic acid is encapsulated by a nanoliposome. In some embodiments, colchicine is conjugated to behenic acid at the acetamide position of a B-ring of colchicine. In some embodiments, the nanoliposome comprises a lipid component comprising DSPC, DOPE, and DSPE-PEG, and further comprises about 5% cholesterol. In some embodiments, colchicine conjugated to behenic acid is present in the nanoliposome in an amount of at least about 400, 500, 600, or more than 600 μg/mL.

In some embodiments, the presently disclosed subject matter also relates to methods for preparing behenic acid conjugated colchicine derivatives. In some embodiments, the methods comprise, consist essentially of, or consist of reacting colchicine with N-dimethyl amino pyridine (DMAP) dissolved in acetonitrile with Boc₂O and triethyl amine at room temperature under argon for at least about 4 hours, optionally 6 hours, optionally wherein the cochicine and DMAP are in equimolar amounts, and relative to the colchicine, the Boc₂O is present in 5-6 equivalents and the triethyl amine is present in 2 equivalents; adding excess Boc₂O and continuing the reaction for about an hour at 80° C. to produce Boc-protected colchicine, optionally wherein the excess Boc₂O added is in an amount of about 0.5-0.6 equivalents; reacting purified Boc-protected colchicine dissolved in methanol with NaOMe in methanol at 4° C. for about 1 hour to produce N-Boc-deacetylcolchicine; reacting purified N-Boc-deacetylcolchicine dissolved in dichloromethane (DCM) with trifluoroacetic acid (TFA) for about 2 hours at room temperature to produce N-deacetylcolchicine; reacting N-deacetylcolchicine with glycolic acid (optionally 1.0 equivalent), NHS, Et₃N, and DIC in DCM for about 24 hours at room temperature to produce colchifoline; and reacting colchifoline with behenic acid in EDC·HCl and DMAP in DCM at room temperature, whereby a behenic acid conjugated colchicine is prepared.

In some embodiments, the presently disclosed subject matter also relates to methods for preparing lipid nanoparticle-encapsulated colchicine derivatives. In some embodiments, the methods comprise, consist essentially of, or consist of encapsulating a colchicine-fatty acid conjugate in a lipid nanoparticle. In some embodiments, the colchicine-fatty acid conjugate is a colchicine-behenic acid conjugate. In some embodiments, the nanoliposome comprises a lipid component comprising DSPC, DOPE, and DSPE-PEG, and further comprises about 5% cholesterol. In some embodiments, the colchicine conjugated to behenic acid is present in the nanoliposome in an amount of at least about 400, 500, 600, or more than 600 μg/mL.

In some embodiments, the presently disclosed subject matter also relates to methods for treating and/or preventing inflammatory and/or cardiovascular diseases, disorders, and/or conditions. In some embodiments, the methods comprise, consist essentially of, or consist of administering to a subject in need thereof an effective amount of a composition as disclosed herein. In some embodiments, the inflammatory disease, disorder, or condition is selected from the group consisting of gout, familial Mediterranean fever, recurrent pericarditis, arthritis, asthma, dermatitis, psoriasis, cystic fibrosis, post transplantation late and chronic solid organ rejection, multiple sclerosis, systemic lupus erythematosus, inflammatory bowel diseases, autoimmune diabetes, diabetic retinopathy, diabetic nephropathy, diabetic vasculopathy, ocular inflammation, uveitis, rhinitis, ischemia-reperfusion injury, post-angioplasty restenosis, chronic obstructive pulmonary disease (COPD), glomerulonephritis, Graves disease, gastrointestinal allergies, conjunctivitis, atherosclerosis, coronary artery disease, angina, and small artery disease. In some embodiments, the cardiovascular disease is selected from the group consisting of Exemplary cardiovascular diseases, including cholesterol- or lipid-related disorders, include, but are not limited to acute coronary syndrome, angina, arteriosclerosis, atherosclerosis, carotid atherosclerosis, cerebrovascular disease, cerebral infarction, congestive heart failure, congenital heart disease, coronary heart disease, coronary artery disease, coronary plaque stabilization, dyslipidemias, dyslipoproteinemias, endothelium dysfunctions, familial hypercholeasterolemia, familial combined hyperlipidemia, hypoalphalipoproteinemia, hypertriglyceridemia, hyperbetalipoproteinemia, hypercholesterolemia, hypertension, hyperlipidemia, intermittent claudication, ischemia, ischemia reperfusion injury, ischemic heart diseases, cardiac ischemia, metabolic syndrome, multi-infarct dementia, myocardial infarction, obesity, peripheral vascular disease, reperfusion injury, restenosis, renal artery atherosclerosis, rheumatic heart disease, stroke, thrombotic disorder, transitory ischemic attacks, and lipoprotein abnormalities associated with Alzheimer's disease, obesity, diabetes mellitus, syndrome X, impotence, multiple sclerosis, Parkinson's disease, and inflammatory diseases.

In some embodiments, the presently disclosed subject matter also provides uses of the presently disclosed compositions for treating and/or preventing inflammatory and/or cardiovascular diseases, disorders, and/or conditions. In some embodiments, the uses comprise, consist essentially of, or consist of administering to a subject in need thereof an effective amount of a composition of the presently disclosed subject matter and/or a combination of different compositions of the presently disclosed subject matter.

Accordingly, it is an object of the presently disclosed subject matter to provide compositions comprising, consisting essentially of, or consisting of colchicine conjugated to behenic acid. This and other objects are achieved in whole or in part by the presently disclosed subject matter.

Further, an object of the presently disclosed subject matter having been stated above, other objects and advantages of the presently disclosed subject matter will become apparent to those skilled in the art after a study of the following description, Figures, and Examples.

### BRIEF DESCRIPTION OF THE FIGURES

FIG. 1. Schematic representation of colchicine-lipid conjugate.

FIG. 2. General synthesis of deactyl colchicine, AI-3-17 (schematic representation and method).

FIGS. 3A and 3B. General synthesis schemes for the production of an exemplary colchicine-behenic acid prodrug, AI-3-25 (FIG. 3A; schematic representation and method) and for production of exemplary colchicine-fatty acyl conjugates generally (FIG. 3B).

FIG. 4. Cryo-EM image of AI-5-25 liposome FIGS. 4A-4F. Dynamic light scattering (DLS) analyses of API colchicine to show size and PDI of the particles with and without cholesterol.

FIG. 5. Drug concentration of AI-5-25 liposomes with 5% cholesterol and without cholesterol.

FIG. 6. Concentration of DLC and colchicine encapsulation in liposomes with and without cholesterol.

FIG. 7. Stability of DLC+5% cholesterol liposomes at the function of the time.

FIG. 8. Release kinetics of DLC+5% cholesterol liposomes at the function of the time. Release kinetics of DLC+5% cholesterol liposomes for 5 days at the function temperature. (A) concentration of the drug remaining in the liposomes (B) release profile of the drugs from liposomes.

FIG. 9. Characteristics and properties of control liposomes and liposomes of the presently disclosed subject matter.

FIG. 10. Stability studies of the liposomes in DMEM, SGF and SIF (A) 0 Hours and (B) 24 Hours

FIG. 11A. A (a) Bright field images of positive control; (b) negative cont (c) RBCs incubated with AI-5-25 liposomes; and (B) hemolysis % of RBCs incubated with three dilutions of five different liposomal system © Photograph of RBCs incubated with three dilutions of five different liposomal system. Triton X 100(+) and 0.9% saline(−) are used as positive and negative controls, respectively.

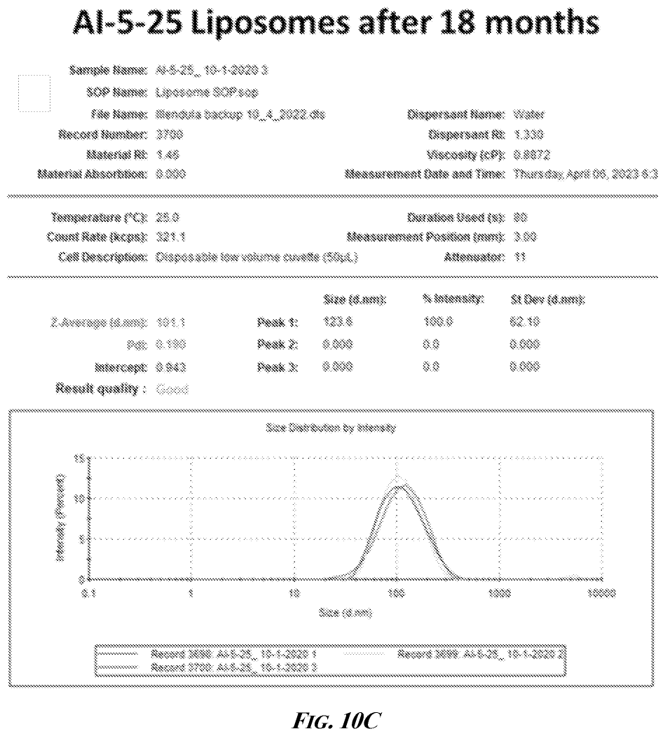

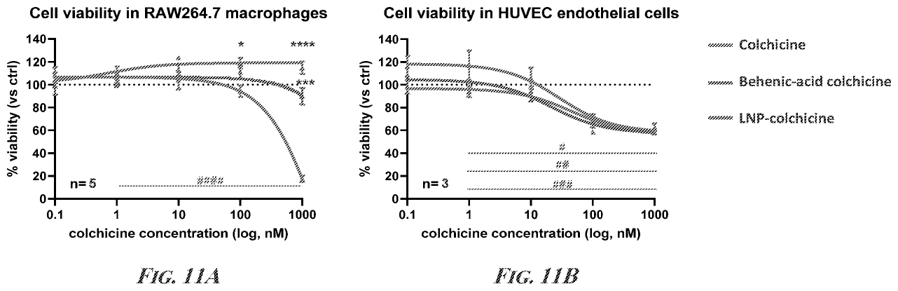

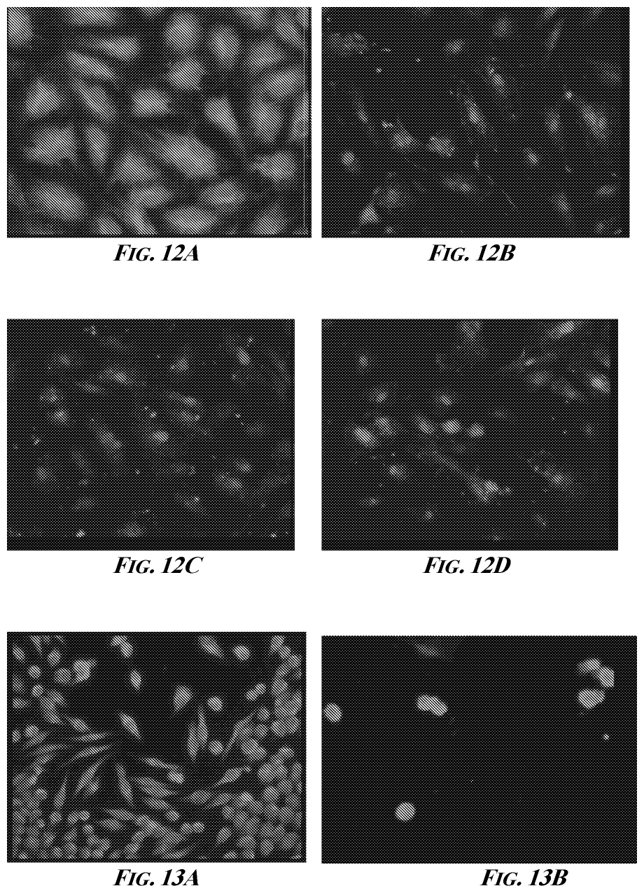

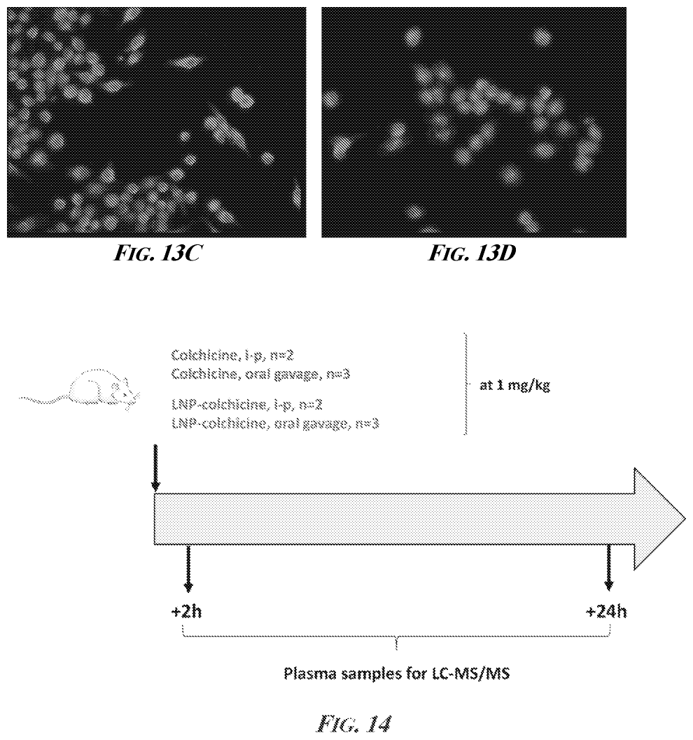

FIGS. 10A-10C. Dynamic light scattering (DLS) analyses of exemplary liposomes of the presently disclosed subject matter.

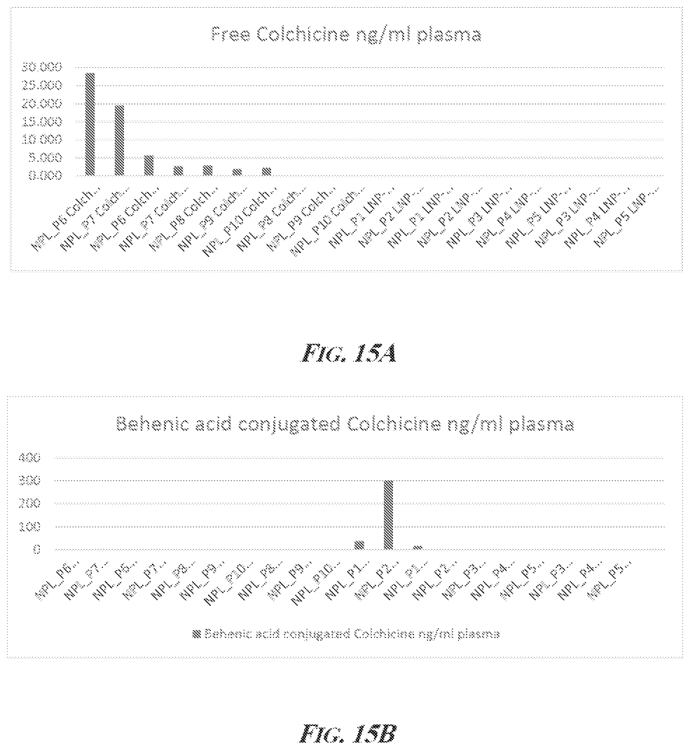

FIGS. 11A and 11B. Cell viability of macrophages (FIG. 11A) and endothelial cells (FIG. 11B), stimulated with three different formulations of colchicine, were evaluated with MTT assay. Cell viability observed with (a) commercialized colchicine; (b) behenic acid-colchicine; (c) liposomes-colchicine. Results are expressed in mean±SEM from 3-5 replicates and statistical analysis was performed using a 2-way-ANOVA, *: p<0.05, ***: p<0.001 vs colchicine; #: p<0.05, ##: p>0.01, ###: p<0.001 vs 0.1 nM of colchicine (green in color version of Figure), behenic-acid colchicine (red in color version of Figure) or liposomes-colchicine (blue in color version of Figure).

FIG. 12. Representation of microtubule polymerization with α/β-tubulin staining of endothelial cells after a 24 hour-stimulation with free media (FIG. 12A), 100 nM colchicine (FIG. 12B), 100 nM behenic-acid colchicine (FIG. 12C), and 100 nM liposomes-colchicine (FIG. 12D).

FIG. 13. Representation of microtubule polymerization with α/β-tubulin staining of J774 macrophages after a 24 hour-stimulation with free media (FIG. 13A), 100 nM colchicine (FIG. 13B), 100 nM behenic-acid colchicine (FIG. 13C), and 100 nM liposomes-colchicine (FIG. 13D).

FIG. 14. Repartition of rats and description of in vivo protocol.

FIGS. 15A and 15B. Analysis of free colchicine in Rat plasma samples (FIG. 15A) and analysis of behenic acid conjugated colchicine in Rat plasma samples (FIG. 15B).

### DETAILED DESCRIPTION

Colchicine, an exemplary hydrophilic drug of the presently disclosed subject matter, is an anti-mitotic and anti-inflammatory drug, which is administered orally. Colchicine has been successfully evaluated in a large-scale clinical trial for mitigation of cardiovascular events, including MI and CVD. Colchicine also suppresses the secretion of cytokines and chemokines, as well as platelet aggregation. However, efficacy is often limited by toxicities and low bioavailability. Colchicine has a narrow therapeutic index and P-glycol-protein (p-gp) drug resistance mechanisms. Colchicine is a class III drug (Biopharmaceutical Classification System), which is characterized by high solubility and low permeability (log P of about 1.5). The administration/delivery of active pharmaceutical ingredients that exhibit these limitations are often enhanced by nanotechnology. The presently disclosed nanoscale formulation offers the potential of enteral delivery, pH-resistance, first-pass-resistance, and arterial targeting of hydrophilic drugs, their precursors, and metabolites thereof including but not limited to colchicine.

This led to the development of the presently disclosed colchicine-fatty acid conjugate (drug-lipid conjugate, DLC) prodrug loaded liposomes. The fatty acid ester linkage is cleaved, forming the active drug. The oleic prodrug itself is novel; the modifications were made to improve colchicine's oral bioavailability. Thus, disclosed herein is a versatile fatty acid-conjugated prodrug to achieve these properties. By way of example and not limitation, a deacetylated colchicine was engineered and synthesized that allowed for the preparation and validation of a colchicine-behenic acid (fatty acid, C22:0) prodrug.

As disclosed herein, the drug-lipid prodrugs helped in the improvement of permeability, stability in the gastric environment (pharmacokinetic properties), and higher drug loading in carriers. A functional group modification at acetamide position in the B-ring of colchicine was chosen, which was not a part of pharmacophore and did not effect tubulin binding activity, to accommodate suitable functional group for lipid conjugation. A hydroxymethyl moiety of colchicine on ring B was appropriate for proposed functional modifications. This modification produced colchifoline, which is a colchicine metabolite and is known for stronger tubulin binding and cytotoxic activity than that of parent moiety colchicine.

This, in some embodiments the presently disclosed subject matter provides engineering and validation of a nanoscale delivery platform to improve pharmacokinetic, pharmacodynamic, and toxicological properties of hydrophilic drugs. To encapsulate hydrophilic colchicine within nanoplatforms, for example, the presently disclosed subject matter provides a proprietary fatty acyl-conjugated colchicine that can be encapsulated within nanoliposomes. It is shown that the exemplary fatty-acyl behenic acid (C22:0)-colchicine prodrug has an improved encapsulation efficiency and release kinetics when encapsulated within nanoliposomes compared with free drugs. This translates to a nanoparticle with improved PK and efficacy.

### I. Definitions

The terminology used herein is for the purpose of describing particular embodiments only and is not intended to be limiting of the presently disclosed subject matter.

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

In describing the presently disclosed subject matter, it will be understood that a number of techniques and steps are disclosed. Each of these has individual benefit and each can also be used in conjunction with one or more, or in some cases all, of the other disclosed techniques.

Accordingly, for the sake of clarity, this description will refrain from repeating every possible combination of the individual steps in an unnecessary fashion. Nevertheless, the specification and claims should be read with the understanding that such combinations are entirely within the scope of the presently disclosed and claimed subject matter.

Following long-standing patent law convention, the terms “a” “an”, and “the” refer to “one or more” when used in this application, including in the claims. For example, the phrase “an antibody” refers to one or more antibodies, including a plurality of the same antibody. Similarly, the phrase “at least one”, when employed herein to refer to an entity, refers to, for example, 1, 2, 3, 4, 5, 6, 7, 8, 9, 10, 15, 20, 25, 30, 35, 40, 45, 50, 75, 100, or more of that entity, including but not limited to whole number values between 1 and 100 and greater than 100.

Unless otherwise indicated, all numbers expressing quantities of ingredients, reaction conditions, and so forth used in the specification and claims are to be understood as being modified in all instances by the term “about”. The term “about”, as used herein when referring to a measurable value such as an amount of mass, weight, time, volume, concentration, or percentage, is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods and/or employ the disclosed compositions. Accordingly, unless indicated to the contrary, the numerical parameters set forth in this specification and attached claims are approximations that can vary depending upon the desired properties sought to be obtained by the presently disclosed subject matter.

A disease or disorder is “alleviated” if the severity of a symptom of the disease, condition, or disorder, or the frequency at which such a symptom is experienced by a subject, or both, are reduced.

As used herein, the term “and/or” when used in the context of a list of entities, refers to the entities being present singly or in combination. Thus, for example, the phrase “A, B, C, and/or D” includes A, B, C, and D individually, but also includes any and all combinations and subcombinations of A, B, C, and D.

The terms “additional therapeutically active compound” and “additional therapeutic agent”, as used in the context of the presently disclosed subject matter, refers to the use or administration of a compound for an additional therapeutic use for a particular injury, disease, or disorder being treated. Such a compound, for example, could include one being used to treat an unrelated disease or disorder, or a disease or disorder which may not be responsive to the primary treatment for the injury, disease, or disorder being treated.

As used herein, the term “adjuvant” refers to a substance that elicits an enhanced immune response when used in combination with a specific antigen.

As used herein, the terms “administration of” and/or “administering” a compound should be understood to refer to providing a compound of the presently disclosed subject matter to a subject in need of treatment.

The term “comprising”, which is synonymous with “including” “containing”, or “characterized by”, is inclusive or open-ended and does not exclude additional, unrecited elements and/or method steps. “Comprising” is a term of art that means that the named elements and/or steps are present, but that other elements and/or steps can be added and still fall within the scope of the relevant subject matter.

As used herein, the phrase “consisting essentially of” limits the scope of the related disclosure or claim to the specified materials and/or steps, plus those that do not materially affect the basic and novel characteristic(s) of the disclosed and/or claimed subject matter. For example, a pharmaceutical composition can “consist essentially of” a pharmaceutically active agent or a plurality of pharmaceutically active agents, which means that the recited pharmaceutically active agent(s) is/are the only pharmaceutically active agent(s) present in the pharmaceutical composition. It is noted, however, that carriers, excipients, and/or other inactive agents can and likely would be present in such a pharmaceutical composition, and are encompassed within the nature of the phrase “consisting essentially of”.

As used herein, the phrase “consisting of” excludes any element, step, or ingredient not specifically recited. It is noted that, when the phrase “consists of” appears in a clause of the body of a claim, rather than immediately following the preamble, it limits only the element set forth in that clause; other elements are not excluded from the claim as a whole.

With respect to the terms “comprising”, “consisting of”, and “consisting essentially of”, where one of these three terms is used herein, the presently disclosed and claimed subject matter can include the use of either of the other two terms. For example, a composition that in some embodiments comprises a given active agent also in some embodiments can consist essentially of that same active agent, and indeed can in some embodiments consist of that same active agent.

As use herein, the terms “administration of” and or “administering” a compound should be understood to mean providing a compound of the presently disclosed subject matter or a prodrug of a compound of the presently disclosed subject matter to a subject in need of treatment.

The term “adult” as used herein, is meant to refer to any non-embryonic or non-juvenile subject. For example, the term “adult adipose tissue stem cell”, refers to an adipose stem cell, other than that obtained from an embryo or juvenile subject.

As used herein, an “agent” is meant to include something being contacted with a cell population to elicit an effect, such as a drug, a protein, a peptide. An “additional therapeutic agent” refers to a drug or other compound used to treat an illness and can include, for example, an antibiotic or a chemotherapeutic agent.

As used herein, an “agonist” is a composition of matter which, when administered to a mammal such as a human, enhances or extends a biological activity attributable to the level or presence of a target compound or molecule of interest in the mammal.

An “antagonist” is a composition of matter which when administered to a mammal such as a human, inhibits a biological activity attributable to the level or presence of a compound or molecule of interest in the mammal.

As used herein, “alleviating a disease or disorder symptom”, means reducing the severity of the symptom or the frequency with which such a symptom is experienced by a patient, or both.

As used herein, an “analog” of a chemical compound is a compound that, by way of example, resembles another in structure but is not necessarily an isomer (e.g., 5-fluorouracil is an analog of thymine).

As used herein, amino acids are represented by the full name thereof, by the three letter code corresponding thereto, and/or by the one-letter code corresponding thereto, as summarized in the following Table:

Amino Acid Codes and Functionally Equivalent Codons

| | 3-Letter | 1-Letter | |
|---|---|---|---|
| Full Name | Code | Code | Functionally Equivalent Codons |
| Aspartic Acid | Asp | D | GAC; GAU |
| Glutamic Acid | Glu | E | GAA; GAG |
| Lysine | Lys | K | AAA; AAG |
| Arginine | Arg | R | AGA; AGG; CGA; CGC; CGG; |
| | | | CGU |
| Histidine | His | H | CAC; CAU |
| Tyrosine | Tyr | Y | UAC; UAU |
| Cysteine | Cys | C | UGC; UGU |
| Asparagine | Asn | N | AAC; AAU |
| Glutamine | Gln | Q | CAA; CAG |
| Serine | Ser | S | ACG; AGU; UCA; UCC; UCG; |
| | | | UCU |
| Threonine | Thr | T | ACA; ACC; ACG; ACU |
| Glycine | Gly | G | GGA; GGC; GGG; GGU |
| Alanine | Ala | A | GCA; GCC; GCG; GCU |
| Valine | Val | V | GUA; GUC; GUG; GUU |
| Leucine | Leu | L | UUA; UUG; CUA; CUC; CUG; |
| | | | CUU |
| Isoleucine | Ile | I | AUA; AUC; AUU |
| Methionine | Met | M | AUG |
| Proline | Pro | P | CCA; CCC; CCG; CCU |
| Phenylalanine | Phe | F | UUC; UUU |
| Tryptophan | Trp | W | UGG |

The expression “amino acid” as used herein is meant to include both natural and synthetic amino acids, and both D and L amino acids. “Standard amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the compositions of the presently disclosed subject matter, and particularly at the carboxy- or amino-terminus, can be modified by methylation, amidation, acetylation or substitution with other chemical groups which can change the peptide's circulating half-life without adversely affecting their activity. Additionally, a disulfide linkage may be present or absent in the compositions of the presently disclosed subject matter.

The term “amino acid” is used interchangeably with “amino acid residue”, and may refer to a free amino acid and to an amino acid residue of a peptide. It will be apparent from the context in which the term is used whether it refers to a free amino acid or a residue of a peptide.

Amino acids have the following general structure:

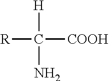

Amino acids may be classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group.

The nomenclature used to describe the peptide compounds of the presently disclosed subject matter follows the conventional practice wherein the amino group is presented to the left and the carboxy group to the right of each amino acid residue. In the formulae representing selected specific embodiments of the presently disclosed subject matter, the amino- and carboxy-terminal groups, although not specifically shown, will be understood to be in the form they would assume at physiologic pH values, unless otherwise specified.

The term “basic” or “positively charged” amino acid as used herein, refers to amino acids in which the R groups have a net positive charge at pH 7.0, and include, but are not limited to, the standard amino acids lysine, arginine, and histidine.

The term “antibody”, as used herein, refers to an immunoglobulin molecule which is able to specifically or selectively bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the presently disclosed subject matter may exist in a variety of forms. The term “antibody” refers to polyclonal and monoclonal antibodies and derivatives thereof (including chimeric, synthesized, humanized and human antibodies), including an entire immunoglobulin or antibody or any functional fragment of an immunoglobulin molecule which binds to the target antigen and or combinations thereof.

Examples of such functional entities include complete antibody molecules, antibody fragments, such as Fᵥ, single chain Fᵥ (scFv), complementarity determining regions (CDRs), V_{L} (light chain variable region), V_{H} (heavy chain variable region), Fab, F(ab′)₂ and any combination of those or any other functional portion of an immunoglobulin peptide capable of binding to target antigen.

Antibodies exist, e.g., as intact immunoglobulins or as a number of well characterized fragments produced by digestion with various peptidases. Thus, for example, pepsin digests an antibody below the disulfide linkages in the hinge region to produce F(ab′)₂ a dimer of Fab which itself is a light chain joined to V_{H}-C_{H1} by a disulfide bond. The F(ab′)₂ may be reduced under mild conditions to break the disulfide linkage in the hinge region, thereby converting the F(ab′)₂ dimer into a Fab₁ monomer. The Fab₁ monomer is essentially an Fab with part of the hinge region (see Paul, 1993). While various antibody fragments are defined in terms of the digestion of an intact antibody, one of skill will appreciate that such fragments may be synthesized de novo either chemically or by utilizing recombinant DNA methodology. Thus, the term antibody, as used herein, also includes antibody fragments either produced by the modification of whole antibodies or those synthesized de novo using recombinant DNA methodologies.

An “antibody heavy chain”, as used herein, refers to the larger of the two types of polypeptide chains present in all antibody molecules.

An “antibody light chain”, as used herein, refers to the smaller of the two types of polypeptide chains present in all antibody molecules.

The term “single chain antibody” refers to an antibody wherein the genetic information encoding the functional fragments of the antibody are located in a single contiguous length of DNA. For a thorough description of single chain antibodies, see Bird et al., 1988; Huston et al., 1988).

By “small interfering RNAs (siRNAs)” is meant, inter alia, an isolated dsRNA molecule comprised of both a sense and an anti-sense strand. In some embodiments, it is greater than 10 nucleotides in length. siRNA also refers to a single transcript which has both the sense and complementary antisense sequences from the target gene, e.g., a hairpin. siRNA further includes any form of dsRNA (proteolytically cleaved products of larger dsRNA, partially purified RNA, essentially pure RNA, synthetic RNA, recombinantly produced RNA) as well as altered RNA that differs from naturally occurring RNA by the addition, deletion, substitution, and/or alteration of one or more nucleotides. RNA interference is a commonly used method to regulate gene expression. This effect is often achieved by using small interfering RNA or short hairpin RNA (shRNA).

The term “humanized” refers to an antibody wherein the constant regions have at least about 80% or greater homology to human immunoglobulin. Additionally, some of the nonhuman, such as murine, variable region amino acid residues can be modified to contain amino acid residues of human origin. Humanized antibodies have been referred to as “reshaped” antibodies. Manipulation of the complementarity-determining regions (CDR) is a way of achieving humanized antibodies. See for example, Jones et al., 1986; Riechmann et al., 1988, both of which are incorporated by reference herein. For a review article concerning humanized antibodies, see Winter & Milstein, 1991, incorporated by reference herein. See also U.S. Pat. Nos. 4,816,567; 5,482,856; 6,479,284; 6,677,436; 7,060,808; 7,906,625; 8,398,980; 8,436,150; 8,796,439; and 10,253,111; and U.S. Patent Application Publication Nos. 2003/0017534, 2018/0298087, 2018/0312588, 2018/0346564, and 2019/0151448, each of which is incorporated by reference in its entirety.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” as used herein is defined as a molecule that provokes an immune response. This immune response may involve either antibody production, or the activation of specific immunologically-competent cells, or both. An antigen can be derived from organisms, subunits of proteins/antigens, killed or inactivated whole cells or lysates.

As used herein, the term “antisense oligonucleotide” or antisense nucleic acid means a nucleic acid polymer, at least a portion of which is complementary to a nucleic acid which is present in a normal cell or in an affected cell. “Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences. The antisense oligonucleotides of the presently disclosed subject matter include, but are not limited to, phosphorothioate oligonucleotides and other modifications of oligonucleotides.

An “aptamer” is a compound that is selected in vitro to bind preferentially to another compound (for example, the identified proteins herein). Often, aptamers are nucleic acids or peptides because random sequences can be readily generated from nucleotides or amino acids (both naturally occurring or synthetically made) in large numbers, but of course they need not be limited to these.

The term “aqueous solution” as used herein can include other ingredients commonly used, such as sodium bicarbonate described herein, and further includes any acid or base solution used to adjust the pH of the aqueous solution while solubilizing a peptide.

The term “binding” refers to the adherence of molecules to one another, such as, but not limited to, enzymes to substrates, ligands to receptors, antibodies to antigens, DNA binding domains of proteins to DNA, and DNA or RNA strands to complementary strands.

“Binding partner”, as used herein, refers to a molecule capable of binding to another molecule.

The term “biocompatible”, as used herein, refers to a material that does not elicit a substantial detrimental response in the host.

As used herein, the terms “biologically active fragment” and “bioactive fragment” of a peptide encompass natural and synthetic portions of a longer peptide or protein that are capable of specific binding to their natural ligand and/or of performing a desired function of a protein, for example, a fragment of a protein of larger peptide which still contains the epitope of interest and is immunogenic.

The term “biological sample”, as used herein, refers to samples obtained from a subject, including but not limited to skin, hair, tissue, blood, plasma, cells, sweat, and urine.

As used herein, the term “chemically conjugated”, or “conjugating chemically” refers to linking one moiety (e.g., a hydrophilic drug and/or a precursor or metabolic product thereof) to a second moiety (e.g., a fatty-acyl group). This linking can occur via one or more covalent bonds created between the first and second moieties using chemical reactions, such as, but not limited to reactions as described herein. Covalent bonds may also be created using a third molecule bridging one moiety to the second moiety. These cross-linkers are able to react with groups, such as but not limited to, amines, sulfhydryls, carbonyls, carbohydrates, esters, and/or carboxylic acids, on one or both moieties. Chemical conjugation also includes non-covalent linkage between a first moiety and a second moiety.

A “coding region” of a gene comprises the nucleotide residues of the coding strand of the gene and the nucleotides of the non-coding strand of the gene which are homologous with or complementary to, respectively, the coding region of an mRNA molecule which is produced by transcription of the gene.

“Complementary” as used herein refers to the broad concept of subunit sequence complementarity between two nucleic acids (e.g., two DNA molecules). When a nucleotide position in both of the molecules is occupied by nucleotides normally capable of base pairing with each other at a given position, the nucleic acids are considered to be complementary to each other at this position. Thus, two nucleic acids are complementary to each other when a substantial number (in some embodiments at least 50%) of corresponding positions in each of the molecules are occupied by nucleotides that can base pair with each other (e.g., A:T and G:C nucleotide pairs). Thus, it is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. By way of example and not limitation, the first region comprises a first portion and the second region comprises a second portion, whereby, when the first and second portions are arranged in an antiparallel fashion, in some embodiments at least about 50%, in some embodiments at least about 75%, in some embodiments at least about 90%, and in some embodiments at least about 95% of the nucleotide residues of the first portion are capable of base pairing with nucleotide residues in the second portion. In some embodiments, all nucleotide residues of the first portion are capable of base pairing with nucleotide residues in the second portion.

A “compound”, as used herein, refers to a polypeptide, an isolated nucleic acid, or other agent used in the method of the presently disclosed subject matter.

A “control” cell, tissue, sample, or subject is a cell, tissue, sample, or subject of the same type as a test cell, tissue, sample, or subject. The control may, for example, be examined at precisely or nearly the same time the test cell, tissue, sample, or subject is examined. The control may also, for example, be examined at a time distant from the time at which the test cell, tissue, sample, or subject is examined, and the results of the examination of the control may be recorded so that the recorded results may be compared with results obtained by examination of a test cell, tissue, sample, or subject. The control may also be obtained from another source or similar source other than the test group or a test subject, where the test sample is obtained from a subject suspected of having a condition, disease, or disorder for which the test is being performed.

A “test” cell is a cell being examined.

As used herein, the term “conservative amino acid substitution” is defined herein as an amino acid exchange within one of the five groups summarized in the following Table:

Exemplary Conservative Amino Acid Substitutions

| Group | Characteristics | Amino Acids |
|---|---|---|
| A. | Small aliphatic, nonpolar, or slightly | Ala, Ser, Thr, Pro, Gly |
| | polar residues | |
| B. | Polar, negatively charged residues and | Asp, Asn, Glu, Gln |
| | their amides | |
| C. | Polar, positively charged residues | His, Arg, Lys |
| D. | Large, aliphatic, nonpolar residues | Met Leu, Ile, Val, Cys |
| E. | Large, aromatic residues | Phe, Tyr, Trp |

A “pathoindicative” cell is a cell that, when present in a tissue, is an indication that the animal in which the tissue is located (or from which the tissue was obtained) is afflicted with a condition, disease, or disorder.

A “pathogenic” cell is a cell that, when present in a tissue, causes, or contributes to a condition, disease, or disorder in the animal in which the tissue is located (or from which the tissue was obtained).

A tissue “normally comprises” a cell if one or more of the cell are present in the tissue in an animal not afflicted with a condition, disease, or disorder.

As used herein, the terms “condition”, “disease condition”, “disease”, “disease state”, and “disorder” refer to physiological states in which diseased cells or cells of interest can be targeted with the compositions of the presently disclosed subject matter. In some embodiments, a disease is cancer, which in some embodiments comprises a solid tumor.

As used herein, the term “diagnosis” refers to detecting a risk or propensity to a condition, disease, or disorder. In any method of diagnosis exist false positives and false negatives. Any one method of diagnosis does not provide 100% accuracy.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, an “effective amount” or “therapeutically effective amount” refers to an amount of a compound or composition sufficient to produce a selected effect, such as but not limited to alleviating symptoms of a condition, disease, or disorder. In the context of administering compounds in the form of a combination, such as multiple compounds, the amount of each compound, when administered in combination with one or more other compounds, may be different from when that compound is administered alone. Thus, an effective amount of a combination of compounds refers collectively to the combination as a whole, although the actual amounts of each compound may vary. The term “more effective” means that the selected effect occurs to a greater extent by one treatment relative to the second treatment to which it is being compared.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA, and mRNA) or a defined sequence of amino acids and the biological properties resulting therefrom. Thus, a gene encodes a protein if transcription and translation of an mRNA corresponding to or derived from that gene produces the protein in a cell or other biological system and/or an in vitro or ex vivo system. Both the coding strand, the nucleotide sequence of which is identical to the mRNA sequence (with the exception of uracil bases presented in the latter) and is usually provided in Sequence Listing, and the non-coding strand, used as the template for transcription of a gene or cDNA, can be referred to as encoding the protein or other product of that gene or cDNA.

The term “epitope” as used herein is defined as small chemical groups on the antigen molecule that can elicit and react with an antibody. An antigen can have one or more epitopes. Most antigens have many epitopes; i.e., they are multivalent. In general, an epitope is roughly five amino acids or sugars in size. One skilled in the art understands that generally the overall three-dimensional structure, rather than the specific linear sequence of the molecule, is the main criterion of antigenic specificity.

As used herein, an “essentially pure” preparation of a particular protein or peptide is a preparation wherein in some embodiments at least about 95% and in some embodiments at least about 99%, by weight, of the protein or peptide in the preparation is the particular protein or peptide.

A “fragment”, “segment”, or “subsequence” is a portion of an amino acid sequence, comprising at least one amino acid, or a portion of a nucleic acid sequence comprising at least one nucleotide. The terms “fragment”, “segment”, and “subsequence” are used interchangeably herein.

As used herein, the term “fragment”, as applied to a protein or peptide, can ordinarily be at least about 3-15 amino acids in length, at least about 15-25 amino acids, at least about 25-50 amino acids in length, at least about 50-75 amino acids in length, at least about 75-100 amino acids in length, and greater than 100 amino acids in length.

As used herein, the term “fragment” as applied to a nucleic acid, may ordinarily be at least about 20 nucleotides in length, typically, at least about 50 nucleotides, more typically, from about 50 to about 100 nucleotides, in some embodiments, at least about 100 to about 200 nucleotides, in some embodiments, at least about 200 nucleotides to about 300 nucleotides, yet in some embodiments, at least about 300 to about 350, in some embodiments, at least about 350 nucleotides to about 500 nucleotides, yet in some embodiments, at least about 500 to about 600, in some embodiments, at least about 600 nucleotides to about 620 nucleotides, yet in some embodiments, at least about 620 to about 650, and most in some embodiments, the nucleic acid fragment will be greater than about 650 nucleotides in length. In the case of a shorter sequence, fragments are shorter.

As used herein, a “functional” biological molecule is a biological molecule in a form in which it exhibits a property by which it can be characterized. A functional enzyme, for example, is one that exhibits the characteristic catalytic activity by which the enzyme can be characterized.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′-ATTGCC-5′ and 3′-TATGGC-5′ share 50% homology.

As used herein, “homology” is used synonymously with “identity”.

The determination of percent identity between two nucleotide or amino acid sequences can be accomplished using a mathematical algorithm. For example, a mathematical algorithm useful for comparing two sequences is the algorithm of Karlin & Altschul, 1990a, modified as in Karlin & Altschul, 1993). This algorithm is incorporated into the NBLAST and XBLAST programs of Altschul et al., 1990a, and can be accessed, for example at the National Center for Biotechnology Information (NCBI) world wide web site. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al., 1997. Alternatively, PSI-Blast or PHI-Blast can be used to perform an iterated search which detects distant relationships between molecules (Altschul et al., 1997) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted.

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the length of the formed hybrid, and the G:C ratio within the nucleic acids.

The term “ingredient” refers to any compound, whether of chemical or biological origin, that can be used in cell culture media to maintain or promote the proliferation, survival, or differentiation of cells. The terms “component”, “nutrient”, “supplement”, and ingredient” can be used interchangeably and are all meant to refer to such compounds. Typical non-limiting ingredients that are used in cell culture media include amino acids, salts, metals, sugars, lipids, nucleic acids, hormones, vitamins, fatty acids, proteins, and the like. Other ingredients that promote or maintain cultivation of cells ex vivo can be selected by those of skill in the art, in accordance with the particular need.

As used herein “injecting”, “applying”, and administering” include administration of a compound of the presently disclosed subject matter by any number of routes and modes including, but not limited to, topical, oral, buccal, intravenous, intramuscular, intra-arterial, intramedullary, intrathecal, intraventricular, transdermal, subcutaneous, intraperitoneal, intranasal, enteral, topical, sublingual, vaginal, ophthalmic, pulmonary, vaginal, and rectal approaches.

Used interchangeably herein are the terms: 1) “isolate” and “select”; and 2) “detect” and “identify”.

The term “isolated”, when used in reference to compositions and cells, refers to a particular composition or cell of interest, or population of cells of interest, at least partially isolated from other cell types or other cellular material with which it naturally occurs in the tissue of origin. A composition or cell sample is “substantially pure” when it is at least 60%, or at least 75%, or at least 90%, and, in certain cases, at least 99% free of materials, compositions, cells other than composition or cells of interest. Purity can be measured by any appropriate method, for example, by fluorescence-activated cell sorting (FACS), or other assays which distinguish cell types. Representative isolation techniques are disclosed herein for antibodies and fragments thereof.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

As used herein, a “ligand” is a compound that specifically or selectively binds to a target compound. A ligand (e.g., an antibody) “specifically binds to”, “is specifically immunoreactive with”, “having a selective binding activity”, “selectively binds to” or “is selectively immunoreactive with” a compound when the ligand functions in a binding reaction which is determinative of the presence of the compound in a sample of heterogeneous compounds. Thus, under designated assay (e.g., immunoassay) conditions, the ligand binds preferentially to a particular compound and does not bind to a significant extent to other compounds present in the sample. For example, an antibody specifically or selectively binds under immunoassay conditions to an antigen bearing an epitope against which the antibody was raised. A variety of immunoassay formats may be used to select antibodies specifically immunoreactive with a particular antigen. For example, solid-phase ELISA immunoassays are routinely used to select monoclonal antibodies specifically immunoreactive with an antigen. See Harlow & Lane, 1988, for a description of immunoassay formats and conditions that can be used to determine specific immunoreactivity.

A “receptor” is a compound that specifically or selectively binds to a ligand.

A ligand or a receptor (e.g., an antibody) “specifically binds to”, “is specifically immunoreactive with”, “having a selective binding activity”, “selectively binds to” or “is selectively immunoreactive with” a compound when the ligand or receptor functions in a binding reaction which is determinative of the presence of the compound in a sample of heterogeneous compounds. Thus, under designated assay (e.g., immunoassay) conditions, the ligand or receptor binds preferentially to a particular compound and does not bind in a significant amount to other compounds present in the sample. For example, a polynucleotide specifically or selectively binds under hybridization conditions to a compound polynucleotide comprising a complementary sequence; an antibody specifically or selectively binds under immunoassay conditions to an antigen bearing an epitope against which the antibody was raised. A variety of immunoassay formats may be used to select antibodies specifically immunoreactive with a particular protein. For example, solid-phase ELISA immunoassays are routinely used to select monoclonal antibodies specifically immunoreactive with a protein. See Harlow & Lane, 1988 for a description of immunoassay formats and conditions that can be used to determine specific or selective immunoreactivity.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule that joins two other molecules either covalently or noncovalently, such as but not limited to through ionic or hydrogen bonds or van der Waals interactions.

The terms “measuring the level of expression” and “determining the level of expression” as used herein refer to any measure or assay which can be used to correlate the results of the assay with the level of expression of a gene or protein of interest. Such assays include measuring the level of mRNA, protein levels, etc. and can be performed by assays such as northern and western blot analyses, binding assays, immunoblots, etc. The level of expression can include rates of expression and can be measured in terms of the actual amount of an mRNA or protein present. Such assays are coupled with processes or systems to store and process information and to help quantify levels, signals, etc. and to digitize the information for use in comparing levels.

The term “modulate”, as used herein, refers to changing the level of an activity, function, or process. The term “modulate” encompasses both inhibiting and stimulating an activity, function, or process. The term “modulate” is used interchangeably with the term “regulate” herein.

The term “nucleic acid” typically refers to large polynucleotides. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil).

As used herein, the term “nucleic acid” encompasses RNA as well as single and double-stranded DNA and cDNA. Furthermore, the terms, “nucleic acid”, “DNA”, “RNA” and similar terms also include nucleic acid analogs, i.e. analogs having other than a phosphodiester backbone. For example, the so-called “peptide nucleic acids”, which are known in the art and have peptide bonds instead of phosphodiester bonds in the backbone, are considered within the scope of the presently disclosed subject matter. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil). Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences”.

The term “nucleic acid construct”, as used herein, encompasses DNA and RNA sequences encoding the particular gene or gene fragment desired, whether obtained by genomic or synthetic methods.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “oligonucleotide” typically refers to short polynucleotides, generally, no greater than about 50 nucleotides. It will be understood that when a nucleotide sequence is represented by a DNA sequence (i.e., A, T, G, C), this also includes an RNA sequence (i.e., A, U, G, C) in which “U” replaces “T”.

The term “otherwise identical sample”, as used herein, refers to a sample similar to a first sample, that is, it is obtained in the same manner from the same subject from the same tissue or fluid, or it refers a similar sample obtained from a different subject. The term “otherwise identical sample from an unaffected subject” refers to a sample obtained from a subject not known to have the disease or disorder being examined. The sample may of course be a standard sample. By analogy, the term “otherwise identical” can also be used regarding regions or tissues in a subject or in an unaffected subject.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

The term “peptide” typically refers to short polypeptides.

The term “pharmaceutical composition” refers to a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

“Pharmaceutically acceptable” means physiologically tolerable, for either human or veterinary application. Similarly, “pharmaceutical compositions” include formulations for human and veterinary use.

As used herein, the term “pharmaceutically acceptable carrier” means a chemical composition with which an appropriate compound or derivative can be combined and which, following the combination, can be used to administer the appropriate compound to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

“Plurality” means at least two.

A “polynucleotide” means a single strand or parallel and anti-parallel strands of a nucleic acid. Thus, a polynucleotide may be either a single-stranded or a double-stranded nucleic acid.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

“Synthetic peptides or polypeptides” refers to non-naturally occurring peptides or polypeptides. Synthetic peptides or polypeptides can be synthesized, for example, using an automated polypeptide synthesizer. Various solid phase peptide synthesis methods are known to those of skill in the art.

The term “prevent”, as used herein, means to stop something from happening, or taking advance measures against something possible or probable from happening. In the context of medicine, “prevention” generally refers to action taken to decrease the chance of getting a disease or condition. It is noted that “prevention” need not be absolute, and thus can occur as a matter of degree.

A “preventive” or “prophylactic” treatment is a treatment administered to a subject who does not exhibit signs, or exhibits only early signs, of a condition, disease, or disorder. A prophylactic or preventative treatment is administered for the purpose of decreasing the risk of developing pathology associated with developing the condition, disease, or disorder.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with, e.g., chromogenic, radioactive, or fluorescent moieties and used as detectable moieties.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a promoter which drives expression of a gene to which it is operably linked, in a constant manner in a cell. By way of example, promoters which drive expression of cellular housekeeping genes are considered to be constitutive promoters.

An “inducible” promoter is a nucleotide sequence which, when operably linked with a polynucleotide which encodes or specifies a gene product, causes the gene product to be produced in a living cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a living cell substantially only if the cell is a cell of the tissue type corresponding to the promoter.

As used herein, “protecting group” with respect to a terminal amino group refers to a terminal amino group of a peptide, which terminal amino group is coupled with any of various amino-terminal protecting groups traditionally employed in peptide synthesis. Such protecting groups include, for example, acyl protecting groups such as formyl, acetyl, benzoyl, trifluoroacetyl, succinyl, and methoxysuccinyl; aromatic urethane protecting groups such as benzyloxycarbonyl; and aliphatic urethane protecting groups, for example, tert-butoxycarbonyl or adamantyloxycarbonyl. See Gross & Mienhofer, 1981 for suitable protecting groups.

As used herein, “protecting group” with respect to a terminal carboxy group refers to a terminal carboxyl group of a peptide, which terminal carboxyl group is coupled with any of various carboxyl-terminal protecting groups. Such protecting groups include, for example, tert-butyl, benzyl, or other acceptable groups linked to the terminal carboxyl group through an ester or ether bond.

The term “protein” typically refers to large polypeptides. Conventional notation is used herein to portray polypeptide sequences: the left-hand end of a polypeptide sequence is the amino-terminus; the right-hand end of a polypeptide sequence is the carboxyl-terminus.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process.

A “highly purified” compound as used herein refers to a compound that is in some embodiments greater than 90% pure, that is in some embodiments greater than 95% pure, and that is in some embodiments greater than 98% pure.

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide may serve a non-coding function (e.g., promoter, origin of replication, ribosome-binding site, etc.) as well.

A host cell that comprises a recombinant polynucleotide is referred to as a “recombinant host cell”. A gene which is expressed in a recombinant host cell wherein the gene comprises a recombinant polynucleotide, produces a “recombinant polypeptide”.

A “recombinant polypeptide” is one which is produced upon expression of a recombinant polynucleotide.

The term “regulate” refers to either stimulating or inhibiting a function or activity of interest.

As used herein, term “regulatory elements” is used interchangeably with “regulatory sequences” and refers to promoters, enhancers, and other expression control elements, or any combination of such elements.

As used herein, the term “secondary antibody” refers to an antibody that binds to the constant region of another antibody (the primary antibody).

As used herein, the term “single chain variable fragment” (scFv) refers to a single chain antibody fragment comprised of a heavy and light chain linked by a peptide linker. In some cases scFv are expressed on the surface of an engineered cell, for the purpose of selecting particular scFv that bind to an antigen of interest.

As used herein, the term “mammal” refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

The term “subject” as used herein refers to a member of species for which treatment and/or prevention of a disease or disorder using the compositions and methods of the presently disclosed subject matter might be desirable. Accordingly, the term “subject” is intended to encompass in some embodiments any member of the Kingdom Animalia including, but not limited to the phylum Chordata (e.g., members of Classes Osteichthyes (bony fish), Amphibia (amphibians), Reptilia (reptiles), Aves (birds), and Mammalia (mammals), and all Orders and Families encompassed therein.

The compositions and methods of the presently disclosed subject matter are particularly useful for warm-blooded vertebrates. Thus, in some embodiments the presently disclosed subject matter concerns mammals and birds. More particularly provided are compositions and methods derived from and/or for use in mammals such as humans and other primates, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), rodents (such as mice, rats, and rabbits), marsupials, and horses. Also provided is the use of the disclosed methods and compositions on birds, including those kinds of birds that are endangered, kept in zoos, as well as fowl, and more particularly domesticated fowl, e.g., poultry, such as turkeys, chickens, ducks, geese, guinea fowl, and the like, as they are also of economic importance to humans. Thus, also provided is the use of the disclosed methods and compositions on livestock, including but not limited to domesticated swine (pigs and hogs), ruminants, horses, poultry, and the like.

As used herein, “substantially homologous amino acid sequences” includes those amino acid sequences which have at least about 95% homology, in some embodiments at least about 96% homology, more in some embodiments at least about 97% homology, in some embodiments at least about 98% homology, and most in some embodiments at least about 99% or more homology to an amino acid sequence of a reference antibody chain. Amino acid sequence similarity or identity can be computed by using the BLASTP and TBLASTN programs which employ the BLAST (basic local alignment search tool) 2.0.14 algorithm. The default settings used for these programs are suitable for identifying substantially similar amino acid sequences for purposes of the presently disclosed subject matter.

“Substantially homologous nucleic acid sequence” means a nucleic acid sequence corresponding to a reference nucleic acid sequence wherein the corresponding sequence encodes a peptide having substantially the same structure and function as the peptide encoded by the reference nucleic acid sequence; e.g., where only changes in amino acids not significantly affecting the peptide function occur. In some embodiments, the substantially identical nucleic acid sequence encodes the peptide encoded by the reference nucleic acid sequence. The percentage of identity between the substantially similar nucleic acid sequence and the reference nucleic acid sequence is at least about 50%, 65%, 75%, 85%, 95%, 99% or more. Substantial identity of nucleic acid sequences can be determined by comparing the sequence identity of two sequences, for example by physical/chemical methods (i.e., hybridization) or by sequence alignment via computer algorithm. Suitable nucleic acid hybridization conditions to determine if a nucleotide sequence is substantially similar to a reference nucleotide sequence are: 7% sodium dodecyl sulfate SDS, 0.5 M NaPO₄, 1 mM EDTA at 50° C. with washing in 2× standard saline citrate (SSC), 0.1% SDS at 50° C.; in some embodiments in 7% (SDS), 0.5 M NaPO₄, 1 mM EDTA at 50° C. with washing in 1×SSC, 0.1% SDS at 50° C.; in some embodiments 7% SDS, 0.5 M NaPO₄, 1 mM EDTA at 50° C. with washing in 0.5×SSC, 0.1% SDS at 50° C.; and more in some embodiments in 7% SDS, 0.5 M NaPO₄, 1 mM EDTA at 50° C. with washing in 0.1×SSC, 0.1% SDS at 65° C. Suitable computer algorithms to determine substantial similarity between two nucleic acid sequences include, GCS program package (Devereux et al., 1984), and the BLASTN or FASTA programs (Altschul et al., 1990a; Altschul et al., 1990b; Altschul et al., 1997). The default settings provided with these programs are suitable for determining substantial similarity of nucleic acid sequences for purposes of the presently disclosed subject matter.

A “sample”, as used herein, refers in some embodiments to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture.

The term “standard”, as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

A “subject” of analysis, diagnosis, or treatment is an animal. Such animals include mammals, in some embodiments, humans.

As used herein, a “subject in need thereof” is a patient, animal, mammal, or human, who will benefit from the method of this presently disclosed subject matter.

The term “substantially pure” describes a compound, e.g., a protein or polypeptide, which has been separated from components which naturally accompany it. Typically, a compound is substantially pure when in some embodiments at least 10%, in some embodiments at least 20%, in some embodiments at least 50%, in some embodiments at least 60%, in some embodiments at least 75%, in some embodiments at least 90%, and in some embodiments at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

The term “symptom”, as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse, and other observers.

A “therapeutic” treatment is a treatment administered to a subject who exhibits signs of pathology for the purpose of diminishing or eliminating those signs.

As used herein, the phrase “therapeutic agent” refers to an agent that is used to, for example, treat, inhibit, prevent, mitigate the effects of, reduce the severity of, reduce the likelihood of developing, slow the progression of, and/or cure, a disease or disorder.

The terms “treatment” and “treating” as used herein refer to both therapeutic treatment and prophylactic or preventative measures, wherein the object is to prevent or slow down (lessen) the targeted pathologic condition, prevent the pathologic condition, pursue or obtain beneficial results, and/or lower the chances of the individual developing a condition, disease, or disorder, even if the treatment is ultimately unsuccessful. Those in need of treatment include those already with the condition as well as those prone to have or predisposed to having a condition, disease, or disorder, or those in whom the condition is to be prevented.

As used herein, the terms “vector”, “cloning vector”, and “expression vector” refer to a vehicle by which a polynucleotide sequence (e.g., a foreign gene) can be introduced into a host cell, so as to transduce and/or transform the host cell in order to promote expression (e.g., transcription and translation) of the introduced sequence. Vectors include plasmids, phages, viruses, etc.

All genes, gene names, and gene products disclosed herein are intended to correspond to homologs and/or orthologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates.

### II. Exemplary Compositions

### II.A. Generally

In some embodiments, the presently disclosed subject matter relates to compositions comprising, consisting essentially of, or consisting of colchicine conjugated to behenic acid, other short chained and unsaturated fatty acids. In some embodiments, the colchicine is conjugated to behenic acid at the acetamide position of a B-ring of colchicine.

In some embodiments, the colchicine conjugated to behenic acid is encapsulated by a delivery vehicle, optionally a liposome, further optionally a nanoliposome. In some embodiments, the nanoliposome comprises a lipid component comprising DSPC, DOPE, and DSPE-PEG, and further comprises about 5% cholesterol. In some embodiments, the inclusion of the about 5% cholesterol enhances the loading of the nanoliposome with the colchicine conjugated to behenic acid as compared to a nanoliposome with a similar lipid component that lacks the cholesterol. Thus, in some embodiments the colchicine conjugated to behenic acid is present in the nanoliposome in an amount of at least about 400, 500, 600, or more than 600 μg/mL.

### II.B. Pharmaceutical Compositions and Administration

The presently disclosed subject matter is also directed to methods of administering the compounds of the presently disclosed subject matter to a subject. Thus, in some embodiments of the presently disclosed subject matter, the compositions are pharmaceutical compositions, optionally pharmaceutical compositions that are pharmaceutically acceptable for use in mammals such as but not limited to humans.

Pharmaceutical compositions comprising the present compounds are administered to a subject in need thereof by any number of routes including, but not limited to, topical, oral, intravenous, intramuscular, intra-arterial, intramedullary, intrathecal, intraventricular, transdermal, subcutaneous, intraperitoneal, intranasal, enteral, topical, sublingual, or rectal means. As such, in some embodiments the presently disclosed compositions are administered by injecting the composition subcutaneously, intraperitoneally, into adipose tissue, and/or intramuscularly into the subject.

In accordance with one embodiment, a method for treating a subject in need of such treatment is provided. The method comprises administering a pharmaceutical composition comprising at least one compound of the presently disclosed subject matter to a subject in need thereof. Compounds identified by the methods of the presently disclosed subject matter can be administered with known compounds or other medications as well.

The pharmaceutical compositions useful for practicing the presently disclosed subject matter may be administered to deliver a dose of between 1 ng/kg/day and 100 mg/kg/day.

The presently disclosed subject matter encompasses the preparation and use of pharmaceutical compositions comprising a compound useful for treatment of the diseases and disorders disclosed herein as an active ingredient. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

As used herein, the term “physiologically acceptable” ester or salt means an ester or salt form of the active ingredient which is compatible with any other ingredients of the pharmaceutical composition, which is not deleterious to the subject to which the composition is to be administered.

The compositions of the presently disclosed subject matter may comprise at least one active peptide, one or more acceptable carriers, and optionally other peptides or therapeutic agents.

For in vivo applications, the compositions of the presently disclosed subject matter may comprise a pharmaceutically acceptable salt. Suitable acids which are capable of forming such salts with the compounds of the presently disclosed subject matter include inorganic acids such as hydrochloric acid, hydrobromic acid, perchloric acid, nitric acid, thiocyanic acid, sulfuric acid, phosphoric acid and the like; and organic acids such as formic acid, acetic acid, propionic acid, glycolic acid, lactic acid, anthranilic acid, cinnamic acid, naphthalene sulfonic acid, sulfanilic acid and the like.

Pharmaceutically acceptable carriers include physiologically tolerable or acceptable diluents, excipients, solvents, or adjuvants. The compositions are in some embodiments sterile and nonpyrogenic. Examples of suitable carriers include, but are not limited to, water, normal saline, dextrose, mannitol, lactose or other sugars, lecithin, albumin, sodium glutamate, cysteine hydrochloride, ethanol, polyols (propylene glycol, polyethylene glycol, glycerol, and the like), vegetable oils (such as olive oil), injectable organic esters such as ethyl oleate, ethoxylated isosteraryl alcohols, polyoxyethylene sorbitol and sorbitan esters, microcrystalline cellulose, aluminum methahydroxide, bentonite, kaolin, agar-agar and tragacanth, or mixtures of these substances, and the like.

In some embodiments wherein a composition of the presently disclosed subject matter is desired to induce an immune response, the compositions of the presently disclosed subject matter can further comprise an adjuvant. In some embodiments, the at least one adjuvant is selected from the group consisting of montanide ISA-51 (Seppic, Inc.), QS-21 (Aquila Pharmaceuticals, Inc.), tetanus helper peptides, GM-CSF, cyclophosamide, bacillus Calmette-Guerin (BCG), corynbacterium parvum, levamisole, azimezone, isoprinisone, dinitrochlorobenezene (DNCB), keyhole limpet hemocyanins (KLH), Freunds adjuvant (complete and incomplete), mineral gels, aluminum hydroxide (Alum), lysolecithin, pluronic polyols, polyanions, peptides, oil emulsions, dinitrophenol, diphtheria toxin (DT).

The pharmaceutical compositions may also contain minor amounts of nontoxic auxiliary pharmaceutical substances or excipients and/or additives, such as wetting agents, emulsifying agents, pH buffering agents, antibacterial and antifungal agents (such as parabens, chlorobutanol, phenol, sorbic acid, and the like). Suitable additives include, but are not limited to, physiologically biocompatible buffers (e.g., tromethamine hydrochloride), additions (e.g., 0.01 to 10 mole percent) of chelants (such as, for example, DTPA or DTPA-bisamide) or calcium chelate complexes (as for example calcium DTPA or CaNaDTPA-bisamide), or, optionally, additions (e.g., 1 to 50 mole percent) of calcium or sodium salts (for example, calcium chloride, calcium ascorbate, calcium gluconate or calcium lactate). If desired, absorption enhancing or delaying agents (such as liposomes, aluminum monostearate, or gelatin) may be used. The compositions can be prepared in conventional forms, either as liquid solutions or suspensions, solid forms suitable for solution or suspension in liquid prior to injection, or as emulsions. Pharmaceutical compositions according to the presently disclosed subject matter can be prepared in a manner fully within the skill of the art.

The compositions of the presently disclosed subject matter, pharmaceutically acceptable salts thereof, or pharmaceutical compositions comprising these compounds may be administered so that the compounds may have a physiological effect. Administration may occur enterally or parenterally; for example, orally, rectally, intracisternally, intravaginally, intraperitoneally, locally (e.g., with powders, ointments, or drops), or as a buccal or nasal spray or aerosol. Parenteral administration is preferred. Particularly preferred parenteral administration methods include intravascular administration (e.g., intravenous bolus injection, intravenous infusion, intra-arterial bolus injection, intra-arterial infusion and catheter instillation into the vasculature), peri- and intra-target tissue injection, subcutaneous injection or deposition including subcutaneous infusion, intramuscular injection, and direct application to the target area, for example by a catheter or other placement device.

Where the administration of the peptide is by injection or direct application, the injection or direct application may be in a single dose or in multiple doses. Where the administration of the compound is by infusion, the infusion may be a single sustained dose over a prolonged period of time or multiple infusions.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

It will be understood by the skilled artisan that such pharmaceutical compositions are generally suitable for administration to animals of all sorts. Subjects to which administration of the pharmaceutical compositions of the presently disclosed subject matter is contemplated include, but are not limited to, humans and other primates, mammals including commercially relevant mammals such as cattle, pigs, horses, sheep, cats, and dogs, birds including commercially relevant birds such as chickens, ducks, geese, and turkeys.

A pharmaceutical composition of the presently disclosed subject matter may be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the presently disclosed subject matter will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the presently disclosed subject matter may further comprise one or more additional pharmaceutically active agents. Particularly contemplated additional agents include anti-emetics and scavengers such as cyanide and cyanate scavengers.

Controlled- or sustained-release formulations of a pharmaceutical composition of the presently disclosed subject matter may be made using conventional technology.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the presently disclosed subject matter are known in the art and described, for example in Gennaro, 1985; Gennaro, 1990; or Gennaro, 2003; each of which is incorporated herein by reference.

Typically, dosages of the compound of the presently disclosed subject matter which may be administered to an animal, in some embodiments a human, range in amount from 1 μg to about 100 g per kilogram of body weight of the animal. While the precise dosage administered will vary depending upon any number of factors, including but not limited to, the type of animal and type of disease state being treated, the age of the animal and the route of administration. In some embodiments, the dosage of the compound will vary from about 1 mg to about 10 g per kilogram of body weight of the animal. In another aspect, the dosage will vary from about 10 mg to about 1 g per kilogram of body weight of the animal.

The compound may be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type of cancer being diagnosed, the type and severity of the condition or disease being treated, the type and age of the animal, etc.

Suitable preparations include injectables, either as liquid solutions or suspensions, however, solid forms suitable for solution in, suspension in, liquid prior to injection, may also be prepared. The preparation may also be emulsified, or the polypeptides encapsulated in liposomes. The active ingredients are often mixed with excipients which are pharmaceutically acceptable and compatible with the active ingredient. Suitable excipients are, for example, water saline, dextrose, glycerol, ethanol, or the like and combinations thereof. In addition, if desired, the vaccine preparation may also include minor amounts of auxiliary substances such as wetting or emulsifying agents, pH buffering agents, and/or adjuvants.

The presently disclosed subject matter also includes a kit comprising the composition of the presently disclosed subject matter and an instructional material which describes administering the composition to a subject. In some embodiments, this kit comprises a (in some embodiments sterile) solvent suitable for dissolving or suspending the composition of the presently disclosed subject matter prior to administering the compound to the subject.

As used herein, an “instructional material” includes a publication, a recording, a diagram, or any other medium of expression which can be used to communicate the usefulness of a composition of the presently disclosed subject matter in the kit for effecting alleviation of the various diseases or disorders recited herein. Optionally, or alternately, the instructional material may describe one or more methods of using the compositions for diagnostic or identification purposes or of alleviation the diseases or disorders in a cell or a tissue of a mammal. The instructional material of the kit of the presently disclosed subject matter may, for example, be affixed to a container which contains a composition of the presently disclosed subject matter or be shipped together with a container which contains the composition.

Alternatively, the instructional material may be shipped separately from the container with the intention that the instructional material and the compound be used cooperatively by the recipient.

The presently disclosed subject matter also related to methods for using the compositions of the presently disclosed subject matter for various purposes. For example, in some embodiments the presently disclosed subject matter also relates to methods for treating and/or preventing diseases, disorders, and/or conditions associated with inflammation.

### II.C. Dosages

An effective dose of a composition of the presently disclosed subject matter is administered to a subject in need thereof. A “treatment effective amount” or a “therapeutic amount” is an amount of a therapeutic composition sufficient to produce a measurable response (e.g., a biologically or clinically relevant response in a subject being treated, such as but not limited to a reduction in scarring and/or fibrosis, particularly as compared to the same subject had the subject not received the composition). Actual dosage levels of active ingredients in the compositions of the presently disclosed subject matter can be varied so as to administer an amount of the active compound(s) that is effective to achieve the desired therapeutic response for a particular subject. The selected dosage level will depend upon the activity of the composition, the route of administration, combination with other drugs or treatments, the severity of the disease, disorder, and/or condition being treated, and the condition and prior medical history of the subject being treated. However, it is within the skill of the art to start doses of the compositions of the presently disclosed subject matter at levels lower than required to achieve the desired therapeutic effect and to gradually increase the dosage until the desired effect is achieved. The potency of a composition can vary, and therefore a “treatment effective amount” can vary. However, using the methods described herein, one skilled in the art can readily assess the potency and efficacy of a composition of the presently disclosed subject matter and adjust the therapeutic regimen accordingly.

After review of the disclosure of the presently disclosed subject matter presented herein, one of ordinary skill in the art can tailor the dosages to an individual subject, taking into account the particular formulation, method of administration to be used with the composition, and particular disease, disorder, and/or condition treated. Further calculations of dose can consider subject height and weight, severity and stage of symptoms, and the presence of additional deleterious physical conditions. Such adjustments or variations, as well as evaluation of when and how to make such adjustments or variations, are well known to those of ordinary skill in the art of medicine.

In some embodiments, a pharmaceutically or therapeutically effective amount of a phototunable hydrogel of the presently disclosed subject matter is administered to a subject at a site of a wound and/or injury, and/or at a site where fibrosis is and/or might occur, and/or at a site where transition of fibroblasts to myofibroblasts would be undesirable.

### II.D. Routes of Administration

Suitable methods for administration of the compositions of the presently disclosed subject matter include, but are not limited to intravenous administration, oral delivery, and delivery directly to a target tissue or organ (e.g., a topical application and/or a site of injury such as but not limited to a muscle injury). Exemplary routes of administration include parenteral, enteral, intravenous, intraarterial, intracardiac, intrapericardial, intraosseal, intracutaneous, subcutaneous, intradermal, subdermal, transdermal, intrathecal, intramuscular, intraperitoneal, intrasternal, parenchymatous, oral, sublingual, buccal, inhalational, and intranasal. The selection of a particular route of administration can be made based at least in part on the nature of the formulation and the ultimate target site where the compositions of the presently disclosed subject matter are desired to act. In some embodiments, the method of administration encompasses features for regionalized delivery or accumulation of the compositions at the site in need of treatment. In some embodiments, the compositions are delivered directly into the site to be treated. By way of example and not limitation, in some embodiments a composition of the presently disclosed subject matter is administered to the subject via a route selected from the group consisting of intraperitoneal, intramuscular, intravenous, and intranasal, or any combination thereof.

The methods described herein use pharmaceutical compositions comprising the molecules described above, together with one or more pharmaceutically acceptable excipients or vehicles, and optionally other therapeutic and/or prophylactic ingredients. Such excipients include liquids such as water, saline, glycerol, polyethyleneglycol, hyaluronic acid, ethanol, cyclodextrins, modified cyclodextrins (i.e., sulfobutyl ether cyclodextrins), etc. Suitable excipients for non-liquid formulations are also known to those of skill in the art. Pharmaceutically acceptable salts can be used in the compositions of the present invention and include, for example, mineral acid salts such as hydrochlorides, hydrobromides, phosphates, sulfates, and the like; and the salts of organic acids such as acetates, propionates, malonates, benzoates, and the like.

Additionally, auxiliary substances, such as wetting or emulsifying agents, biological buffering substances, surfactants, and the like, may be present in such vehicles. A biological buffer can be virtually any solution which is pharmacologically acceptable and which provides the formulation with the desired pH, i.e., a pH in the physiologically acceptable range. Examples of buffer solutions include saline, phosphate buffered saline, Tris buffered saline, Hank's buffered saline, and the like.

Depending on the intended mode of administration, the pharmaceutical compositions may be in the form of a liquid, suspension, cream, ointment, lotion, or the like, preferably in unit dosage form suitable for single administration of a precise dosage. The compositions can in some embodiments include one or more pharmaceutically acceptable carriers and, in addition, may include other pharmaceutical agents, adjuvants, diluents, buffers, etc.

In some embodiments, the mode of administration is a liquid form, which can then be cured by application of light of the appropriate wavelength, intensity, and duration to cure the phototunable hydrogels of the presently disclosed subject matter at a site of interest.

### Iii. Methods for Making and Using the Presently Disclosed Compositions

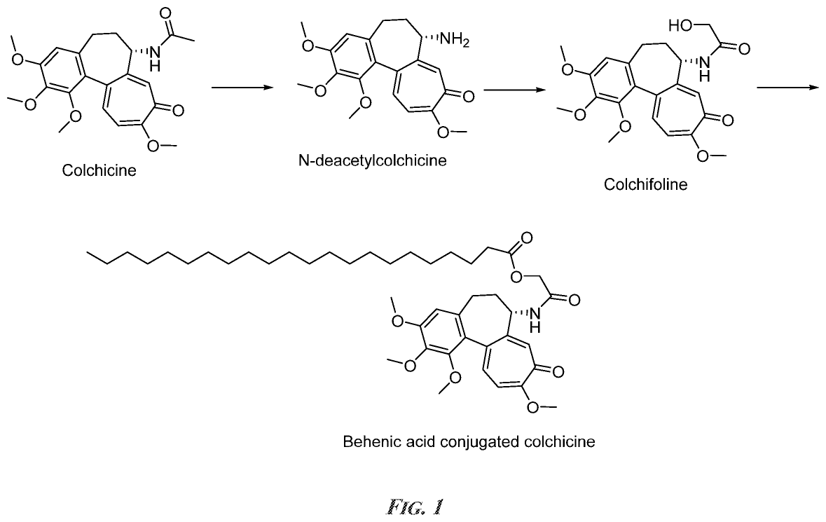

In some embodiments, the presently disclosed subject matter also relates to methods for preparing a lipid nanoparticle-encapsulated colchicine derivative. Generalized strategies for preparing fatty-acid derivatives of colchicine are shown in FIGS. 1-3.

In some embodiments, the methods comprise, consist essentially of, or consist of encapsulating a colchicine-fatty acid conjugate as disclosed herein in a lipid nanoparticle. In some embodiments, the colchicine-fatty acid conjugate is a colchicine-behenic acid conjugate. In some embodiments, the nanoliposome comprises a lipid component comprising DSPC, DOPE, and DSPE-PEG, and further comprises about 5% cholesterol. In some embodiments, the colchicine conjugated to behenic acid is present in the nanoliposome in an amount of at least about 400, 500, 600, or more than 600 μg/mL.

The presently disclosed subject matter also relates in some embodiments to methods for using the presently disclosed compositions for treating and/or preventing inflammatory and/or cardiovascular diseases, disorders, and/or conditions. In some embodiments, the methods comprise, consist essentially of, or consist of administering to a subject in need thereof an effective amount of a composition as disclosed herein.

Any inflammatory and/or cardiovascular diseases, disorders, and/or conditions for which treatment with colchicine per se might be appropriate can be treated with the presently disclosed compositions. By way of example and not limitation, in some embodiments the inflammatory disease, disorder, or condition is selected from the group consisting of gout, familial Mediterranean fever, recurrent pericarditis, arthritis, asthma, dermatitis, psoriasis, cystic fibrosis, post transplantation late and chronic solid organ rejection, multiple sclerosis, systemic lupus erythematosus, inflammatory bowel diseases, autoimmune diabetes, diabetic retinopathy, diabetic nephropathy, diabetic vasculopathy, ocular inflammation, uveitis, rhinitis, ischemia-reperfusion injury, post-angioplasty restenosis, chronic obstructive pulmonary disease (COPD), glomerulonephritis, Graves disease, gastrointestinal allergies, conjunctivitis, atherosclerosis, coronary artery disease, angina, and small artery disease. Similarly and also by way of example and not limitation, in some embodiments the cardiovascular disease, disorder, or condition is selected from the group consisting of cholesterol- or lipid-related disorders, include, but are not limited to acute coronary syndrome, angina, arteriosclerosis, atherosclerosis, carotid atherosclerosis, cerebrovascular disease, cerebral infarction, congestive heart failure, congenital heart disease, coronary heart disease, coronary artery disease, coronary plaque stabilization, dyslipidemias, dyslipoproteinemias, endothelium dysfunctions, familial hypercholeasterolemia, familial combined hyperlipidemia, hypoalphalipoproteinemia, hypertriglyceridemia, hyperbetalipoproteinemia, hypercholesterolemia, hypertension, hyperlipidemia, intermittent claudication, ischemia, ischemia reperfusion injury, ischemic heart diseases, cardiac ischemia, metabolic syndrome, multi-infarct dementia, myocardial infarction, obesity, peripheral vascular disease, reperfusion injury, restenosis, renal artery atherosclerosis, rheumatic heart disease, stroke, thrombotic disorder, transitory ischemic attacks, and lipoprotein abnormalities associated with Alzheimer's disease, obesity, diabetes mellitus, syndrome X, impotence, multiple sclerosis, Parkinson's disease, and inflammatory diseases.

Thus, in some embodiments the presently disclosed subject matter relates to uses of the presently disclosed compositions, including but not limited to compositions comprising, consisting essentially of, or consisting of modified colchicine-containing nanoliposomes for treating and/or preventing diseases, disorders, and/or conditions associated with inflammation and/or cardiovascular disease in subjects.

Additionally, in some embodiments, the chemically modified colchicine DLCs can be loaded into tunable liposomes. As used herein, a “tunable liposome” is a liposome for which through modifications of the liposomal synthesis parameters, starting material molar ratios, and initial drug concentration, the physiochemical properties of the liposomes (size, surface charge, drug concentration, etc.) can be selected for individually. also PEG chain lengths and other common NP surface modifications such as ligand functionalization for targeting (antibodies, aptamers, peptide sequences, etc.) and “stealth” (PEG, CD-47, albumin, etc.) can be adorned and biologically active on the NP surface.

It is noted that hydrophilic drugs have certain limitations, like drug leakage, burst release, and very low encapsulation, which was seen in the case of colchicine when loaded in the liposomes, Hence by increasing the lipophilicity of the drug with the modifications disclosed herein, the entrapment of the drug can be increased, altering the release profiles of the drugs as well, which in some embodiments can result in sustained release and/or avoiding burst or uncontrolled release.

For example, colchicine is an orally absorbed drug, and its main route of absorption is to cross the intestinal membrane. Oral drugs undergo passive diffusion at the tight junctions between the intestinal enterocytes or through the lipid cell membrane. Conjugating the fatty acids to low-permeability drugs can improve their lipophilicity. Changing the fatty acid carbon chain length and unsaturation alters the lipophilicity of the drugs, thus altering the permeability. Moreover, the hydrolysis rate of the prodrugs depends on the chain length of the fatty acid. As a result, prodrug molecules are in some embodiments hydrolyzed more slowly as the fatty acid chain length increases because of the protective effect of the ester bond on the bulky substituent group.

### EXAMPLES

The following EXAMPLES provide illustrative embodiments. In light of the present disclosure and the general level of skill in the art, those of skill will appreciate that the following EXAMPLES are intended to be exemplary only and that numerous changes, modifications, and alterations can be employed without departing from the scope of the presently disclosed subject matter.

Without further description, it is believed that one of ordinary skill in the art can, using the preceding description and the following illustrative EXAMPLES, make and utilize the compounds of the presently disclosed subject matter and practice the methods of the presently disclosed subject matter. The following EXAMPLES therefore particularly point out embodiments of the presently disclosed subject matter and are not to be construed as limiting in any way the remainder of the disclosure.

### Example 1

### Synthesis of a De-Acetylated Colchicine

The synthesis of deacetyl colchicine is achieved in three steps, and the method was optimized after multiple iterations for scale-up. Colchicine is an alkaloid and is derived from the plant Colchicum autumnale. FDA approved this natural product in 1961 for the management of gout and Familial Mediterranean Fever (FMF). Direct chemical modifications are limited on the colchicine molecule. It has a 7-membered C-ring, and its tropolone ether moiety is sensitive to chemical modifications and easily oxidizable. Acetamides are quite stable compounds. Hydrolysis is facilitated under heating with strong acids/bases, and C—N bond cleavage is challenging with common hydrolysis agents, especially in the presence of other labile functional groups, methoxy (OMe). The protection of N-acetyl, N-formyl and N-benzoyl undergo hydrolysis under milder conditions.

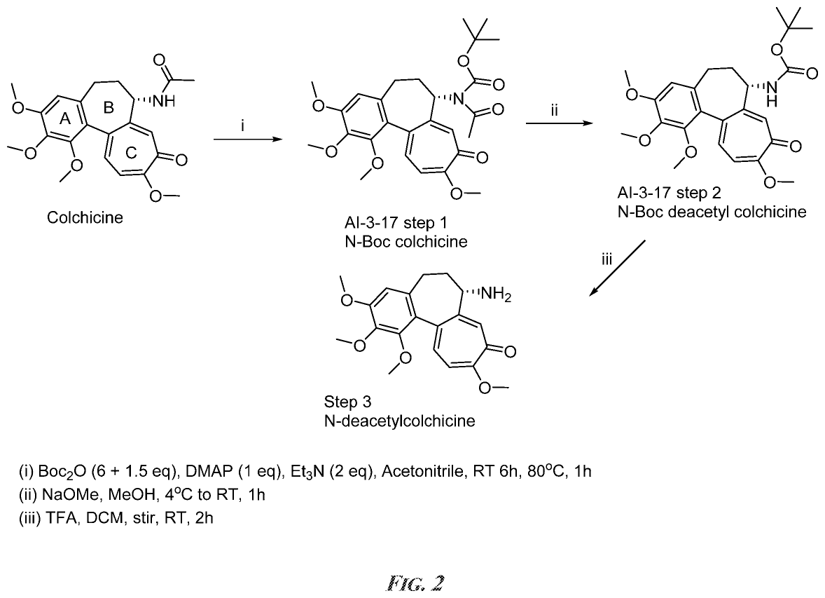

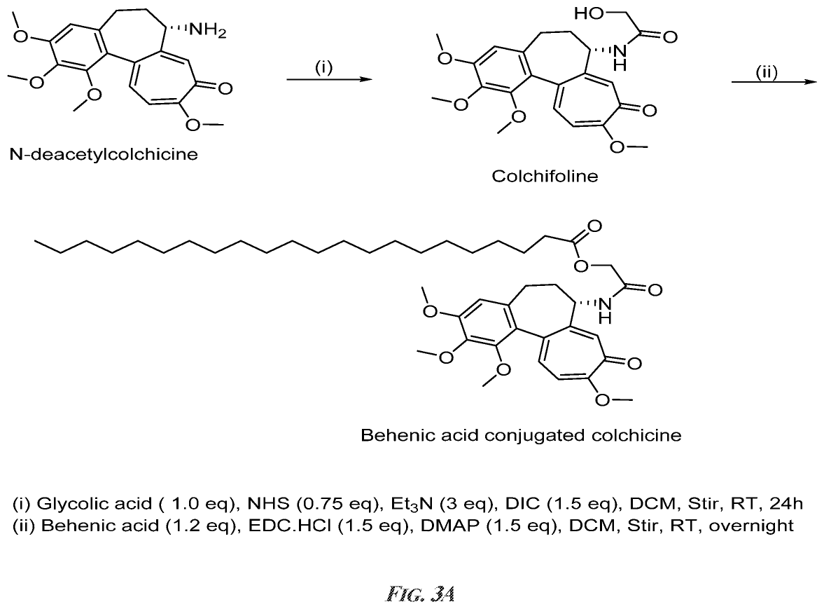

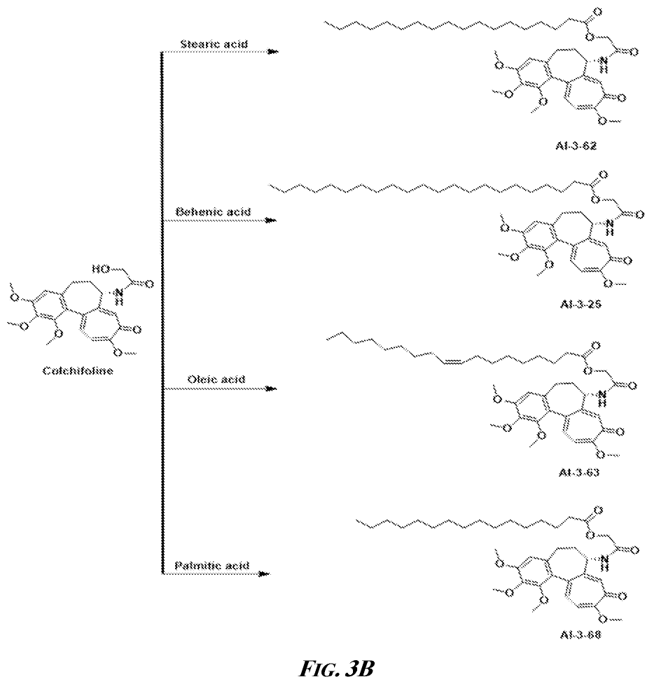

To address the issues associated with the synthesis of N-Boc protected colchicine, poor yield, and byproducts, was considered by modulating the temperature (heating 80-100° C. vs. RT), adding excess equivalents of reagent (Boc anhydride) and the nature of heat (conventional, microwave). N-Boc protected colchicine in the process of the synthesis of the deacetyl colchicine as reported in the literature. However, in most cases they focused on the product isolation and did not discuss yield, the effect of temperature on the byproduct formation and other side products. Flash chromatography was utilized, which provided an advantage over the gravity column and mass directed HPLC purification in the separation of the byproduct. A generalized scheme for preparing N-deacetylcolchicine in three steps is provided in FIG. 2.

Synthesis of N-Boc-colchicine (Step 1): Equimolar (2.5 mmol) ratio of colchicine and N, and N-dimethyl amino pyridine (DMAP) were dissolved in Acetonitrile (10 mL). Boc₂O (15 mmol, 6 eq) and triethyl amine (5 mmol, 2 eq) were added to the above mixture and stirred at RT under argon for 6 hours, and excess of Boc₂O (1.5 mmol, 0.6 eq) was added and heated at 80° C. for 1 hour. The usual work-up was performed to isolate the reaction mixture and purified by Buchi's Pure C-815 flash. It yielded pure Boc-protected colchicine (65%). The product AI-3-17 (Step-1) characterized by ¹H NMR and ESI mass confirmed the product formation and carried to the next step.

Synthesis of N-Boc-deacetylcolchicine (Step 2): N-Boc-colchicine, from the Step-1 (1.5 mmol) was dissolved in MeOH (8 mL), and NaOMe in MeOH (2 mL, 25% w/v) was added to the above reaction mixture at 4° C. for 1 hour. The completion of the reaction was monitored by thin layer chromatography (TLC) and quenched with saturated ammonium chloride solution, followed by usual work-up yielded reaction mixture, which is carried to next step without further purification.

Synthesis of N-deacetylcolchicine (Step 3): N-Boc-deacetyl colchicine from Step-2 was dissolved in dichloromethane (DCM) and TFA (trifluoroacetic acid) was added drop-wise at 4° C. Reaction mixture was stirred at RT for 2 hours. The completion of the reaction was monitored by TLC, and saturated sodium carbonate solution was added slowly to quench the reaction, followed by usual work-up yielded reaction mixture, which was purified by flash chromatography to yield pure deacetyl colchicine. The product, AI-3-17, was characterized by NMR and ESI mass.

A colchicine-behenic acid prodrug was then synthesized as set forth in FIG. 3 and is described in more detail herein below. Synthesis of a colchicine-fatty acid prodrug was achieved in two steps. Synthesis of colchifoline, a hydroxyacetyl colchicine, was synthesized in improved yields. Colchifoline AI-3-21 was used in the synthesis of colchicine-fatty acid prodrug by our proprietary method using environmentally friendly, water soluble, coupling reagents.

Synthesis of N-(2-hydroxyacetyl) colchicine, Colchifoline: Equimolar ratio of AI-3-17, N-deacetyl colchicine (0.42 mmol) and glycolic acid (0.42 mmol) and Et₃N (1.2 mmol, 3 eq), N-hydroxysuccinimide (0.31 mmol, 0.75 eq) were dissolved in DCM (8 mL). DIC (0.63 mmol, 1.5 eq) was added to the above reaction mixture and stirred overnight under argon at RT. Added excess 1 more equivalent of reagents after checking the TLC and stirred for 3 more hours. After usual work-up, the reaction mixture was purified to remove the excess reagents. The 1NMR of the product showed the presence of DIC urea and the purification process was repeated to yield pure product in 50% yield. NMR and ESI Mass spectral data confirmed the structure of the product, AI-3-21.

Synthesis of Colchicine-behenic acid prodrug: Behenic acid (C22:0) (0.18 mmol, 1.2 eq), EDC·HCl (0.23 mmol, 1.5 eq) and DMAP (0.23 mmol, 1.5 eq) were dissolved in DCM (3 mL) and colchifoline, AI-3-21, (0.15 mmol, 1 eq) in DCM added to the above reaction mixture and stirred under argon at RT overnight. The reaction was followed by TLC and indicated the absence of starting material. After usual work-up, the reaction mixture was purified by flash chromatography to yield pure compound in 55% yield. The ESI mass (M+) of the compound indicates the presence of the prodrug. Characterization of the product by NMR (1H, 13C) and CHN analysis and high resolution Mass is performed. The pure compound AI-3-25 is utilized in the targeted liposome formulation preparation.

Oleic acid, stearic acid, and palmitic acid conjugates of colchicine were also prepared in a similar procedure. Instead of behenic acid, oleic acid (C18:1; 0.18 mmol, 1.2 eq) was employed to prepare the colchicine-oleic acid conjugate, stearic acid (C18:0; 0.18 mmol, 1.2 eq) was employed to prepare a colchicine-stearic acid conjugate, and palmitic acid (C16:0; 0.18 mmol, 1.2 eq) was employed to prepare a colchicine-palmitic acid conjugate. The yield of a pure compound was 40.81%, 40.65%, and 57.08% for colchicine-oleic acid, colchicine-stearic acid, and colchicine-palmitic acid conjugates, respectively. The ESI mass (M+) of the compounds indicated the presence of the prodrugs. The characterization of the product by NMR (¹H, ¹³C), CHN analysis, and high-resolution mass spectroscopic analysis is also performed. The pure compounds, such as AI-3-62, AI-3-63, and AI-3-68 can be utilized in the targeted liposome formulation preparation.

FIG. 3B presents a schematic representation of the synthesized prodrugs with colchicine and fatty acids.

### Example 2

### Synthesis and Characterization of a Liposome that Intercalates Behenic-Acid Bioconjugated Colchicine (LDC Liposomes)

AI-3-25, the LDC, was described above and this LDC was utilized in the development of nanoliposomes and compared with non-conjugated, commercially available, colchicine liposomes. Colchicine drug from Oakwood chemicals was utilized in the preparation of the liposomes and labeled as API Colchicine liposomes.

### Example 3

### Synthesis of Liposomal Colchicine

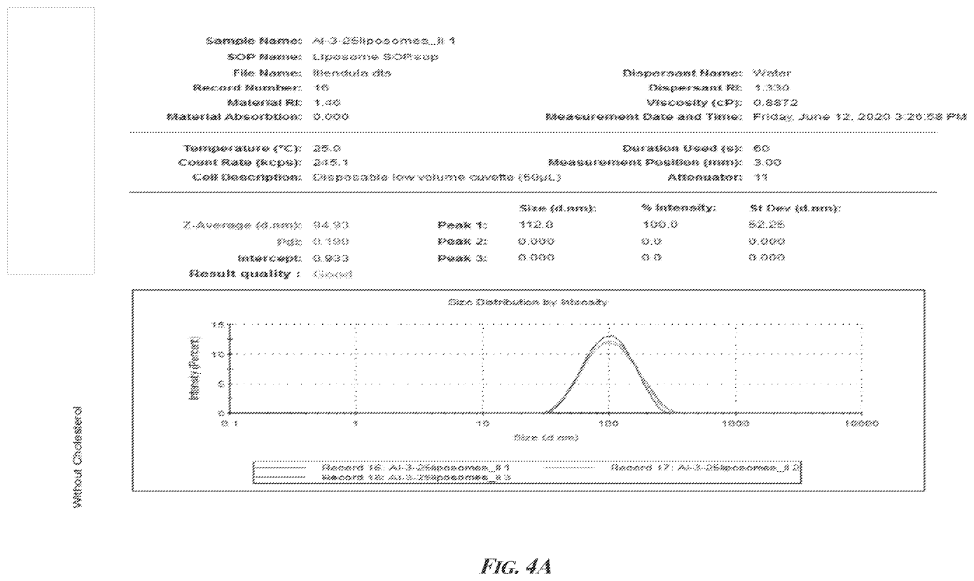

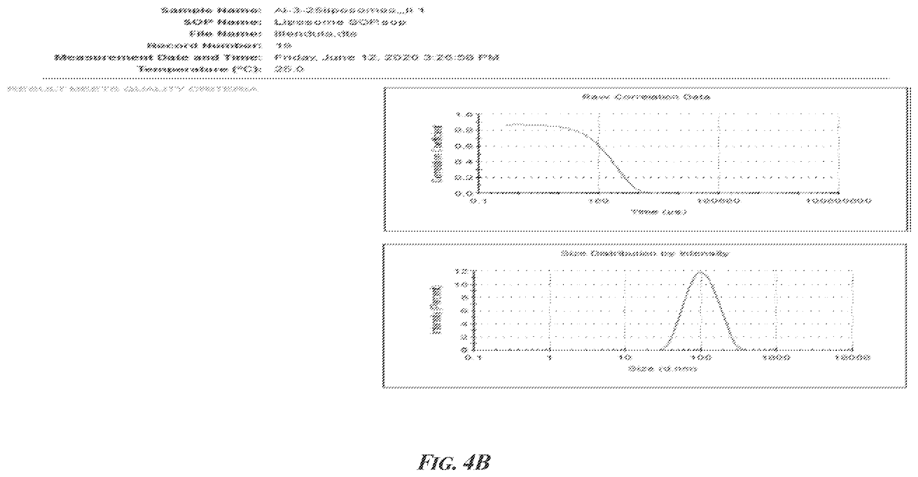

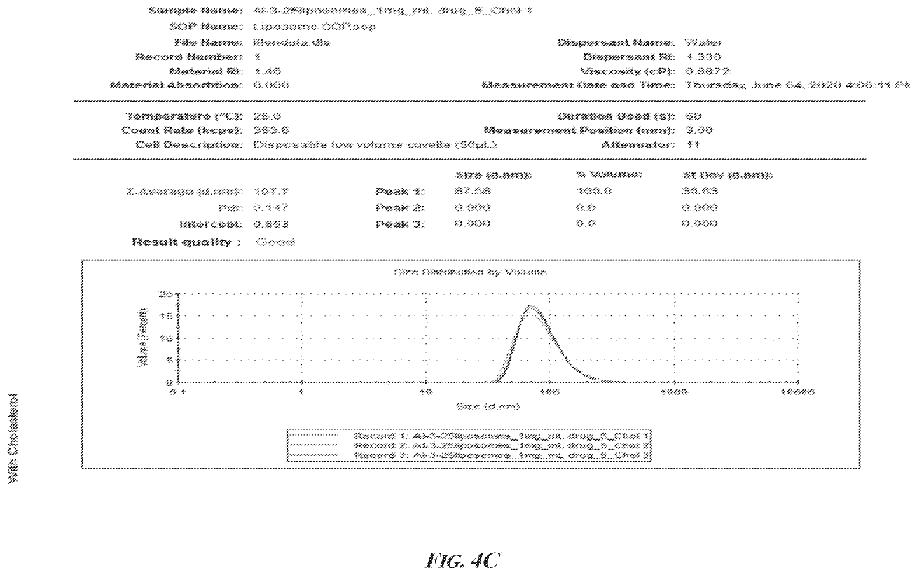

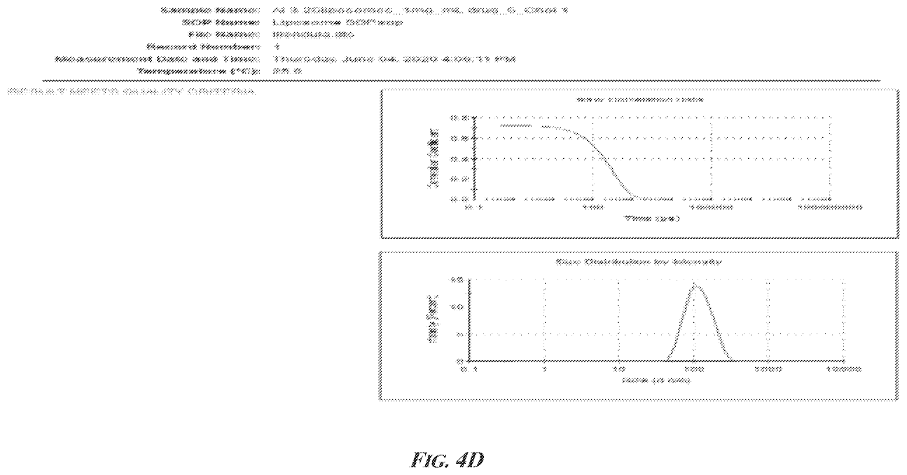

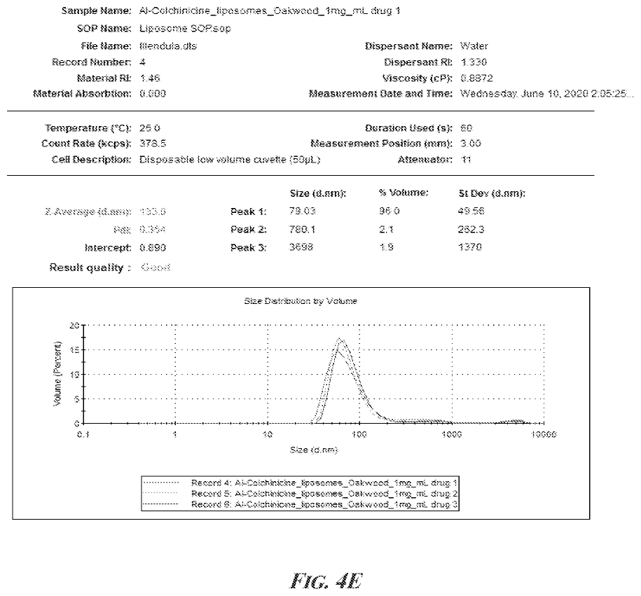

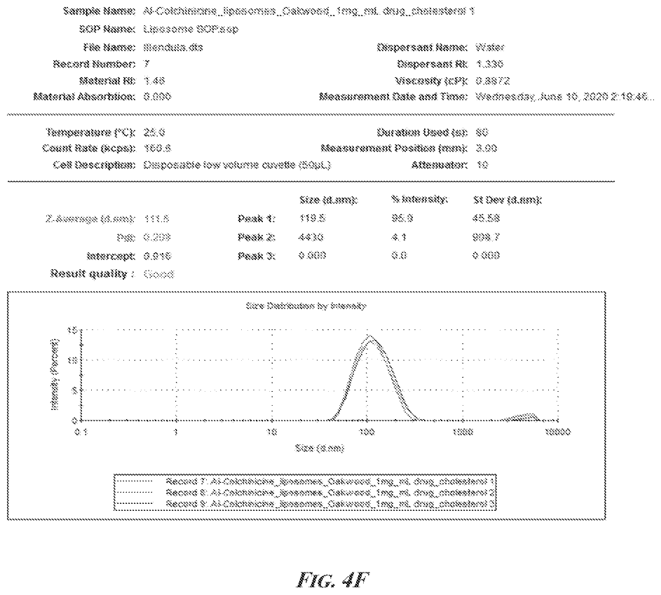

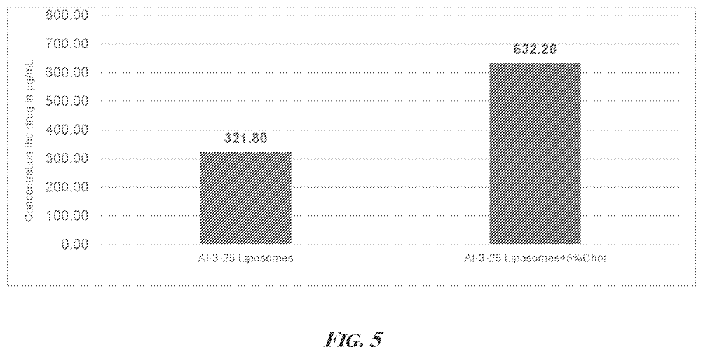

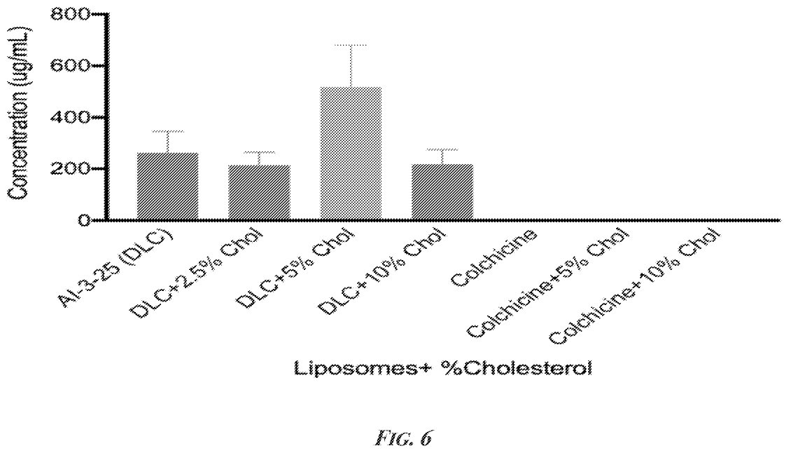

Highly pegylated nanoliposomes that encapsulate therapeutic doses of the LDC were synthesized by our proprietary methods. Optimization of encapsulation of the LDCs was evaluated by mass spectrometry as a function of passive trapping methodologies as well as molar ratios of DSPC, DOPE, and DSPE-PEG pegylated lipids. Brief description of the method; the lipids were combined in specific molar ratios of 5.6, 2.8, 1.0 respectively, total volume of 1 mL and DLC in chloroform at concentration of 1 mg/mL. The lipid mixture was dried under a stream of nitrogen, and hydrated above the lipid transition temperature, with sterile phosphate buffered saline and sonicated until the lipids were suspended in the solution. For small-scale manufacturing, the lipid solution was then extruded 11 times through a 100 nm polycarbonate membrane at temperature above the lipid transition temperature using a temperature controlled water bath. Formulations generated with each loading method was purified using a Sepharose CL-B4 column to separate a free drug from encapsulated liposomes. The morphology of the liposomes were observed using Cryo-EM, the sizes of the liposomes were around 100 nm as shown in FIG. 4. Hydrodynamic size (diameter) of the liposome samples were measured in aqueous solutions using dynamic light scattering (DLS) at 25° C. This measurement included the intensity-weighted average diameter overall size populations (Z-avg), the polydispersity index (PDI), the volume-weighted average diameter over the major volume peak (Vol-Peak), and its percentage of the total population (Vol-Peak % Vol). A Malvern Zetasizer Nano was used for these measurements in batch mode. The z-Average size and PDI of the particles without cholesterol is about 94.93 nm and PDI is 0.19 and with cholesterol is 107.7 nm and PDI 0.14. Both samples displayed similar profiles based upon average size distribution by intensity (see FIGS. 4A-4F). The drug concentration in the liposomes and the reproducibility of manufacturing liposomal LDCs at scale was quantified by LC-MS. LC-MS was performed using a C18 separation column with acetonitrile or 10 v/o methanol/water on an Agilent 1100 system LC system coupled to ABI 4100. These procedures have previously been used by the Kester Laboratory to assess the concentration of drugs in liposomal formulations and calibration curves to assess 5 ng/mL-50 mg/mL of each compound have been established. The drug concentration in the liposomes prepared by using 5% cholesterol is almost double when compared with liposomes without cholesterol (FIG. 5). The optimization of drug concentration in liposomes was achieved by varying the cholesterol concentration (FIG. 6).

### Example 4

### QA/QC Stability and Release Kinetic Studies for Behenic Acid-Conjugated Colchicine Nanoparticles

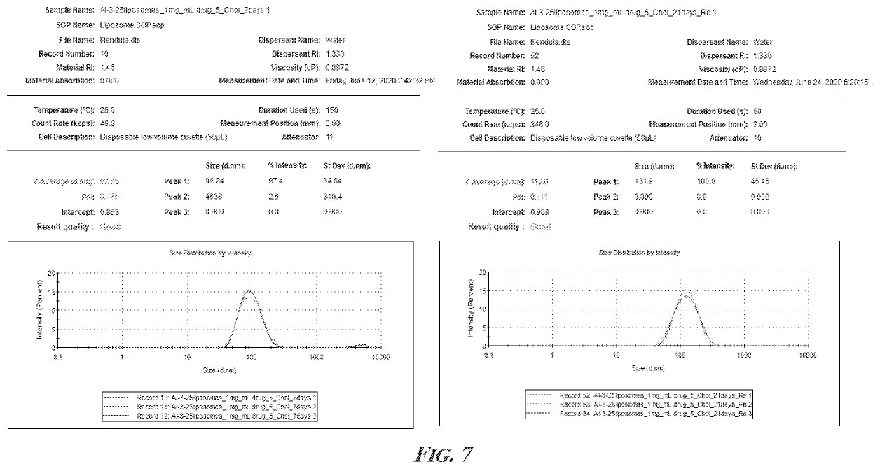

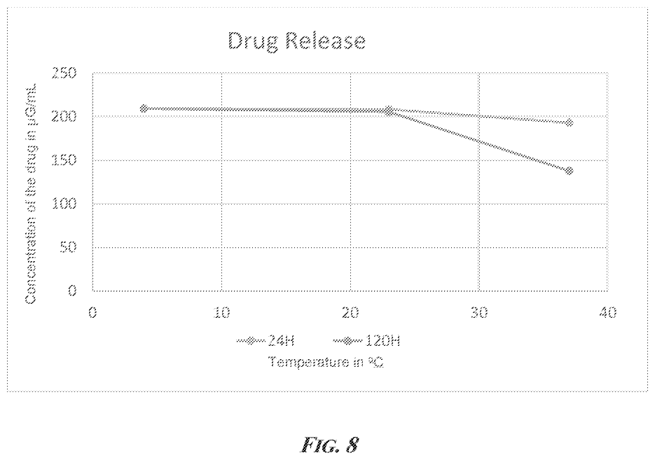

The long-term storage stability was determined by diluting the stock solution (stored at 4° C.) periodically and measuring hydrodynamic size, using a Malvern Zetasizer Nano as described above. The DLS graph (FIG. 7) shows the stability of the particles at 4° C. at the function of the time. The formulation of day 1 and day 21 DLS data was included whereas DLS of 50, 90 days were recorded to follow-up on the stability at 4° C. The stability studies for drug release kinetics was conducted on the optimized batch with PBS at 4° C., RT and 37° C. at 24 and 120 hours (FIG. 8).

### Example 5

### Scale-Up of Colchicine-Behenic Acid Conjugate, AI-3-25

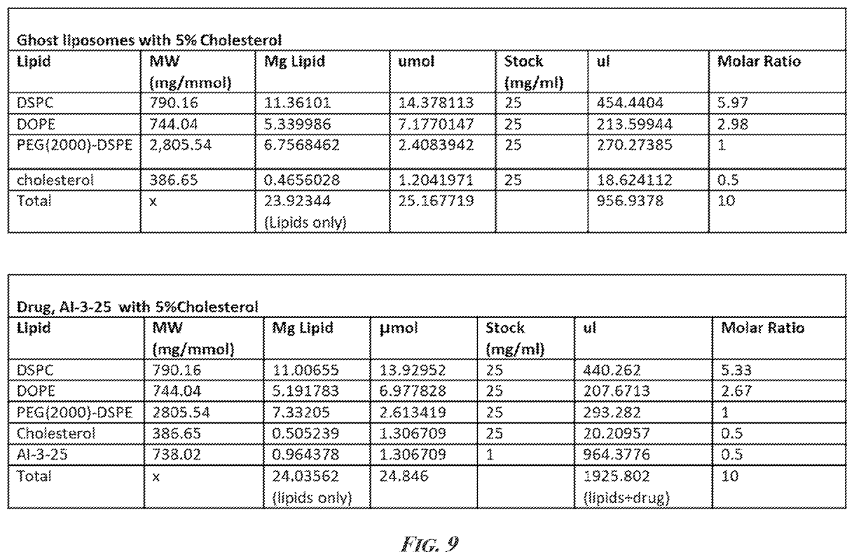

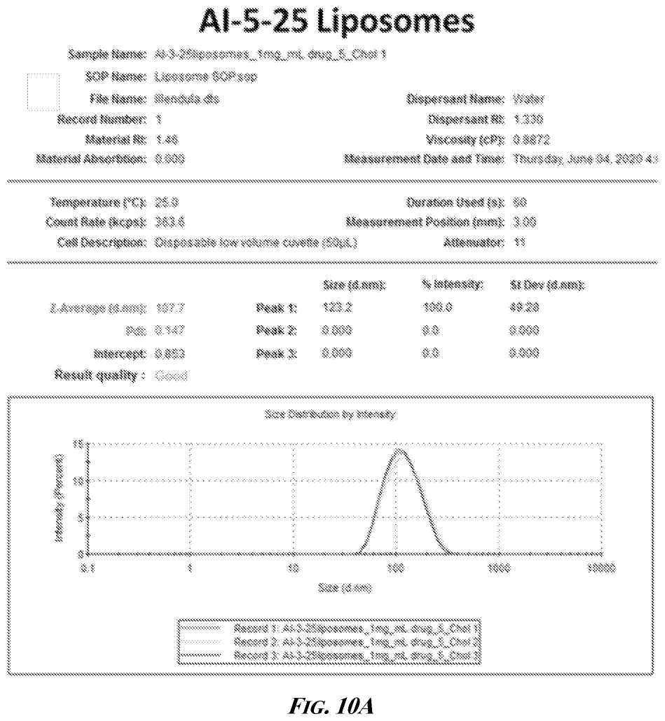

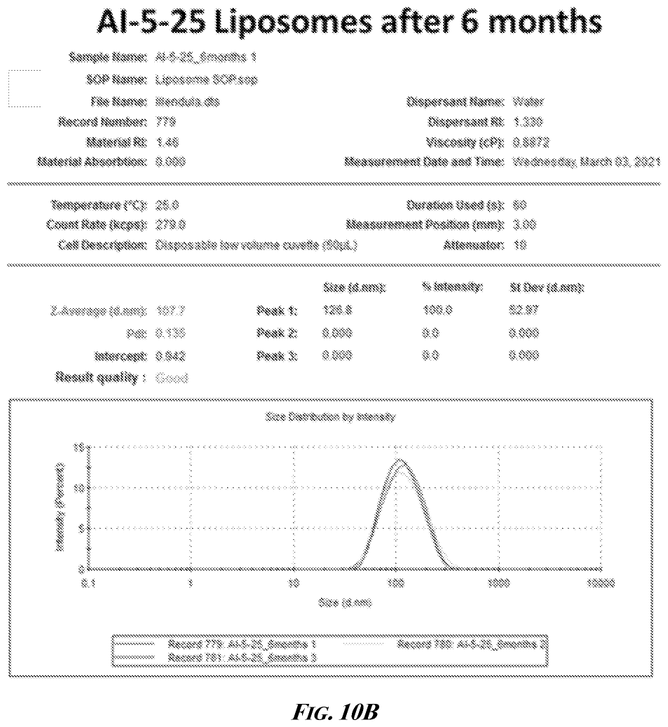

As described above, the synthesis of behenic acid-conjugated colchicine, AI-3-25, was achieved from colchifoline, which was derived from deacetyl colchicine. Liposome formulation of colchicine-lipid conjugate, AI-5-25 has been developed containing: 1,2 distearoyl-sn-glycero-3-phosphocholine (DSPC); 1,2-dioleoyl-sn-glycero-3-phosphoethanolamine (DOPE); 1,2-distearoyl-sn-glycero-3-phosphoethanolamine-N-[methoxy(polyethylene glycol)-2000](ammonium salt) (PEG(2000)-DSPE) and cholesterol. All lipid components were purchased from Avanti Polar Lipids, Inc. All lipid and drug, behenic acid-conjugated colchicine, AI-3-25, stock solutions were prepared in chloroform. Ghost liposomes are prepared by same method without a drug. See FIG. 9 for a summary of the properties of the ghost liposomes and the AI-5-25 containing liposomes.

The lipids mixtures thoroughly mixed in a glass test tube and then the chloroform was evaporated under nitrogen at 35-40° C. to complete dryness (forms a thin film around the test tube) for 2-3 hrs. Trace amounts of chloroform were removed under reduced pressure via rotary evaporator for 30 minutes. The liposomes were rehydrated with 1×PBS in a heat shaker for 2 hours (60° C.; 600 rpm), vortexing every 15 minutes. The Liposomes were then sized using an Avanti Mini extruder (Avanti Polar Lipids) fitted with a 0.1 μm polycarbonate membrane. This is achieved by passing the liposome mixture through the extruder back and forth 13 times.

The sized liposomes were purified by a gravity exclusion column with Sepharose CL-2B beads (Sigma Aldrich) and eluted with 1×PBS to separated liposomes from the free drug. The liposomes thus collected are stored at 4° C. in 1×PBS. Liposomes were characterized by dynamic light scattering (DLS) after dilution with 1×PBS (20 μL+1×PBS 480 μL). The average size is approximately 100 nm with PDI in the range of 0.14 to 0.18. The following DLS data showed the stability of liposomes after 6 months and 18 months at 4° C. The particles were stable, homogenous in size, and without any agglomeration (FIGS. 10A-10C).

### Example 6

### Stability Studies of the Liposomes

Colchicine absorbs after oral administration in the gastrointestinal tract, therefore gastrointestinal stability of prodrug liposomes is a concern in the development of nanoparticles for oral delivery. The stability of liposomes was evaluated in a simulated gastric fluid (SGF) and simulated intestinal fluid (SIF) without enzymes at room temperature for a period of 24 h. The stability studies were performed on colchicine conjugated behenic acid-liposomes (AI-5-25), colchicine conjugated oleic acid-liposomes (AI-5-51-I), colchicine conjugated stearic acid-liposomes (AI-5-51-II), colchicine conjugated palmitic acid-liposomes (AI-5-53) and colchifoline loaded liposomes (AI-5-55). No significant change in the particle size was observed during 24 h as shown in the FIG. 10, indicating the high stability of nanoparticles under the gastrointestinal conditions for all the liposomes. In addition, these liposomes were found to remain stable in DMEM medium containing 10% fetal bovine serum (FBS) for 24 h. For the drug release kinetic study, dialysis tubing (molecular weight cutoff=12000 Da) was incubated in 20 mL of PBS overnight at 4° C. and washed. The dialysis tubes were sealed with liposomes and dialysed against SGF for 1 hour followed by 20 mL simulated intestinal fluid (SIF) for 3 h and finally 20 mL PBS buffer for the total 120 h, at 37° C. with gentle shaking. The concentrations of the drugs in the dialysis buffer were measured using mass spectroscopy at the time 1, 2, 4, 8, 12, 24, 36, 48, 72, 96 and 120 h, respectively.

### Example 7

### Hemolysis Assay

To study the biocompatibility of the colchine-prodrug loaded liposomes and the prodrugs, we performed a hemolysis assay experiment. Red blood cells (RBCs) were collected from a mice using EDTA syringe. Briefly, the blood samples were diluted with 0.9% saline and centrifuged at 2000 rpm for 10 min to isolate the RBCs from the serum. RBCs were then washed with 0.9% saline until a colorless supernatant is obtained. The purified RBCs were diluted with 5 mL of 0.9% of saline. Prior to this, three different dilutions of each colchicine-prodrug liposomes were diluted with 0.9% saline. From each liposomal system 100 μL, 50 μL and 25 μL of the liposomal solution was diluted to 0.7 mL as shown in FIG. 11C(a). Further, 0.3 mL of diluted RBCs was mixed with 0.7 mL of the diluted liposomal solution, which were kept at 37° C. for 2 h. Herein, 1% v/v Triton X-100 and 0.9% saline solutions were used as positive and negative controls, respectively. Finally, the mixtures were centrifuged at 2000 rpm for 5 min, and 150 μL of supernatant from all the samples was added to a 96-well plate. The absorbance values of the supernatants were measured at 540 nm using a Cytation3 microplate reader. The hemolysis percentage of RBCs was calculated by using the following formula: Haemolysis %=(sample absorbance−negative control absorbance/positive control absorbance−negative control absorbance)×100. The hemolytic percentages of all the liposome dilutions are illustrated in FIG. 11B. The structural destruction of the RBCs were observed using a fluorescence microscope and 60× objective in the bright field mode (FIG. 11C (a-c)). The bright field image (FIG. 11C(a)) of the positive control showed clearly that the RBCs were ruptured. Besides, the RBCs retained its structures when treated with AI-5-25 and 100 μL dilution were similar to the negative control as shown in FIG. 11C (b-c). Our results suggested that none of the formulations resulted in hemolysis. They are safe and completely non hemolytic as % hemolysis is less than 5%, rendering them as clinically favorable.

### Example 8

### In Vitro Biological Assays

### Concentration-Dependent Cell Toxicity of Free Colchicine but not Liposomes Colchicine

Cell toxicity was tested on RAW264.7 macrophages and HUVEC endothelial cells. All cells were plated on 96-wells plate (2000 and 8000 cells per well, respectively) and exposed to colchicine or controls three days after (FIGS. 11A and 11B). Cells were exposed to commercialized colchicine (#9754, Sigma-Aldrich), behenic-acid colchicine or liposomal nanoparticles-colchicine (liposomes-colchicine, generated by the nanoSTAR Institute) as well as their respective controls (free media, ethanol/DMSO, ghost liposomes) from 0.1 to 10000 nM. After 24 hours of stimulation, media was removed and replaced with MTT solution (M2128, Sigma-Aldrich) at 0.375 mg/mL for 4 hours at 37° C. Viable cells produced dark blue formazan products. These products were dissolved in an isopropanol/Triton X-100, 10%/HCl solution, and read at 575 nm for absorbance density values to determine the cell viability. The percentage of the viable cells was calculated using the following formula: (%)=[100×(colchicine compound absorbance)/(own control absorbance)].

As expected, a 24 hour stimulation with free colchicine at high concentration decreased cell viability in both macrophages and endothelial cells. In macrophages, behenic acid colchicine (no liposome) and liposomes-colchicine did not induce any toxicity and actually slightly improved cell viability, especially for concentrations greater than 100 nM. In endothelial cells, the three formulations of colchicine induced similar response on cells, even though their was a statistical increase in viability at lower doses with liposomes-colchicine.

### Example 9

### Colchicine Liposome Alters Microtubule Polymerization and Cell Shape Similarly to Free Colchicine or Behenic Acid-Conjugated Colchicine

The efficiency of colchicine to bind microtubules and alter their polymerization was evaluated on HUVEC endothelial cells (FIG. 12) and J774 macrophages (FIG. 13). Cells were plated, respectively, at 5000 and 10000 cells/cm2 three days before colchicine stimulation. Cells were exposed to commercialized colchicine (#9754, Sigma-Aldrich), behenic-acid colchicine or liposomal nanoparticles-colchicine (liposome-colchicine), and their respective controls: free media, ethanol/DMSO, free liposomes; at equal concentration (100 nM). After 24 hours of stimulation, cells were fixed with paraformaldehyde 4% for 10 minutes. Blocking was performed with 10% normal goat serum with 0.2% Triton X-100. The immunocytometry staining used an α/β-tubulin antibody (#2148, Cell Signaling, 1/100, overnight, +4° C.) followed with a goat anti-rabbit antibody Alexa 555 (#A21429, Invitrogen, 1/200, 1 hour, room temperature). DAPI was used for nuclear staining. Pictures at 20× magnification were taken using AxioVision software SE64 4.9.1 (Zeiss).

Filamentous microtubule arrays were observed in both endothelial cells and macrophages treated with control media. At 100 nM. free colchicine significantly dysregulated microtubule polymerization, cell division and cell shape in endothelial cells and in macrophages). At 100 nM behenic-acid colchicine and AI-5-25-treated cells showed similar modifications to free colchicine in endothelial cells and in macrophages.

### Example 10

### In Vivo Pharmacokinetics

The route of administration and the PK profile for liposomes colchicine were evaluated. The scaled-up formulation of liposomes colchicine, 15 mL, ghost formulation, non-liposomal behenic acid-conjugated colchicine and free colchicine were evaluated to determine the PK profile of liposomes colchicine, as a function of route of administration, intra-peritoneal injection and oral gavage. This preliminary in vivo study is designed to estimate PK parameters of liposomes colchicine and mode of administration of the liposomes. This study was performed using 10 rats divided into 4 groups as specified in FIG. 14. Rats received colchicine or liposomes-colchicine at 1 mg/kg by intra-peritoneal injection or oral gavage. Two and 24 hours after administration, blood was collected for a quantification of plasmatic colchicine and liposomes by liquid chromatography-mass spectrometry (LC-MS/MS). After 2 days, one of the rats was sacrificed because of difficult breathing and lethargic condition. No severe toxicities were observed after liposomes-colchicine administration, by either IP or gavage. All plasma samples were analyzed for colchicine and colchicine-behenic acid conjugate.

LC/MS samples were prepared as per standard plasma extraction protocol. All samples were analyzed against API colchicine and behenic acid conjugated colchicine as shown in FIGS. 15A and 15B.

The LC/MS data clearly indicates the presence of behenic acid conjugated colchicine in plasma at 2 hours by IP, with diminished levels by gavage. While free colchicine as a metabolite is not found in plasma of rats that were treated with behenic acid conjugated colchicine, colchifoline, our intermediate was found.

### Example 11

### Spectral Data of Exemplary Conjugates

AI-3-25: Colchicine+BA: Prodrug: ¹H NMR (600 MHz, CDCl₃) δ 7.37 (s, 1H), 7.31 (d, J=10.7 Hz, 1H), 6.88 (dd, J₁=9.2, J₂=5.8 Hz, 1H), 6.83 (d, J=11.1 Hz, 1H), 6.53 (s, 1H), 4.69 (d, J=11.8 Hz, 1H), 4.58 (d, J=15.7 Hz, 1H), 4.50 (d, J=15.7 Hz, 1H), 4.00 (s, 3H), 3.94 (d, J=1.4 Hz, 3H), 3.90 (s, 3H), 3.65 (d, J=2.8 Hz, 3H), 2.57-2.45 (m, 2H), 2.43-2.37 (m, 2H), 2.29-2.23 (m, 1H), 1.91-1.82 (m, 1H), 1.69-1.61 (m, 2H), 1.25 (s, 36H), 0.88 (t, J=7.0 Hz, 3H).

AI-3-62: Colchicine+SA: Prodrug: ¹H NMR (600 MHz, CDCl₃) δ 7.43 (s, 1H), 7.32 (d, J=10.7 Hz, 1H), 6.84 (d, J=10.9 Hz, 1H), 6.54 (s, 1H), 4.70 (dd, J₁=6.6 Hz, J₂=5.2 Hz, 1H), 4.56 (dd, J₁=35.2 Hz, J₂=15.6 Hz, 2H), 4.00 (s, 3H), 3.95 (s, 3H), 3.91 (s, 3H), 3.65 (s, 3H), 2.54 (dd, J₁=13.5 Hz, J₂=6.3 Hz, 1H), 2.46-2.32 (m, 4H), 2.31-2.26 (m, 1H), 2.04-1.97 (m, 1H), 1.91 (td, J₁=11.9 Hz, J₂=6.8 Hz, 1H), 1.62 (dd, J₁=14.7 Hz, J₂=7.3 Hz, 2H), 1.25 (s, 26H), 0.88 (d, J=6.9 Hz, 3H).

AI-3-63: Colchicine+OA: Prodrug: ¹H NMR (600 MHz, CDCl₃) δ 7.45 (d, J=3.9 Hz, 1H), 7.32 (d, J=10.7 Hz, 1H), 6.85 (d, J=10.8 Hz, 1H), 6.54 (s, 1H), 5.34 (dd, J₁=4.0 Hz, J₂=1.5 Hz, 2H), 4.69 (tt, J₁=12.4 Hz, J₂=6.2 Hz, 1H), 4.56 (dd, J₁=36.8 Hz, J₂=15.6 Hz, 2H), 4.00 (s, 3H), 3.94 (s, 3H), 3.91 (s, 3H), 3.65 (s, 3H), 2.58-2.50 (m, 1H), 2.40 (dt, J₁=7.4 Hz, J₂=6.5 Hz, 2H), 2.28 (dt, J₁=12.6 Hz, J₂=6.5 Hz, 2H), 2.01 (dd, J₁=12.5 Hz, J₂=6.5 Hz, 4H), 1.90 (d, J=6.9 Hz, 1H), 1.66-1.58 (m, 2H), 1.31-1.26 (m, 20H), 0.87 (t, J=3.6 Hz, 3H).

AI-3-68: Colchicine+PA: Prodrug: ¹H NMR (600 MHz, CDCl₃) δ 7.37 (d, J=4.0 Hz, 1H), 7.31 (d, J=10.6 Hz, 1H), 6.90 (s, 1H, NH, br), 6.83 (d, J=10.8 Hz, 1H), 6.53 (s, 1H), 4.73-4.64 (m, 1H), 4.58 (d, J=15.7 Hz, 1H), 4.50 (d, J=15.7 Hz, 1H), 4.00 (s, 3H), 3.94 (s, 3H), 3.90 (s, 3H), 3.65 (s, 3H), 2.58-2.52 (m, 1H), 2.42 (dd, J=14.0, 6.9 Hz, 2H), 2.30-2.23 (m, 1H), 1.67-1.63 (m, 2H), 1.26 (s, 24H), 1.14 (d, J=6.5 Hz, 2H), 0.88 (s, 3H).

### Discussion of the Examples

A goal of the presently disclosed subject matter was to engineer and validate a nanoscale delivery platform to improve pharmacokinetic, pharmacodynamics and toxicological properties. To encapsulate hydrophillic colchicine within a nanoplatform, the presently disclosed subject matter relates in some embodiments to fatty acyl-conjugated colchicines that can be encapsulated within nanoliposomes. The presently disclosed fatty-acyl (Behenic acid, C22:0) colchicine derivative has an improved encapsulation efficiency and improved release kinetics when encapsulated within nanoliposomes as compared with free drug. This translates to a nanoparticle with improved PK and efficacy.

As such, drug-lipid conjugates (DLCs) improve membrane permeability, stability in the gastric environment (pharmacokinetic properties), and higher drug loading in nanocarriers. An exemplary functional group modification at the acetamide position in the B-ring of colchicine, which is not a part of the pharmacophore and does not affect tubulin binding activity, is exemplified herein to accommodate a suitable functional group for lipid conjugation. A hydroxymethyl moiety of colchifoline on ring B is appropriate for the proposed functional modifications. This modification produces colchifoline, a colchicine metabolite known for stronger tubulin binding and cytotoxicity as compared with colchicine.

In summary, disclosed herein are behenic acid conjugated-colchicine liposomes synthesized by a highly effective method, which offers optimal encapsulation efficiency, whereas encapsulation of free colchicine was not successful. The data presented herein established that modification of drug by lipid conjugation offered high retention of the drug and encapsulation efficiency (EE %) of 45%-50% whereas API's EE % is almost zero. The results presented herein strongly support that the presently disclosed approach, encapsulation of a colchicine-fatty acid conjugate, was highly effective for colchicine drug nanoformulation to improve the PK profile and to reduce the toxicity of API. These results, including optimized scale-up strongly supported the broader utility of this technology by conjugating hydrophilic drugs with hydrophobic moieties, improving drug retention in liposomes with higher encapsulation efficiency, and improved PK.

### REFERENCES

All references listed in the instant disclosure, including but not limited to all patents, patent applications and publications thereof, scientific journal articles, and database entries (including but not limited to UniProt, EMBL, and GENBANK® biosequence database entries and including all annotations available therein) are incorporated herein by reference in their entireties to the extent that they supplement, explain, provide a background for, and/or teach methodology, techniques, and/or compositions employed herein. The discussion of the references is intended merely to summarize the assertions made by their authors. No admission is made that any reference (or a portion of any reference) is relevant prior art. Applicants reserve the right to challenge the accuracy and pertinence of any cited reference.
Abouaitah et al. (2020) Targeted nano-drug delivery of colchicine against colon cancer cells by means of mesoporous silica nanoparticles. Cancers 12(1):1-30. https://doi.org/10.3390/cancers12010144
Altschul et al. (1990a) Basic local alignment search tool. J Mol Biol 215:403-410.
Altschul et al. (1990b) Protein database searches for multiple alignments. Proc Natl Acad Sci USA 87:14:5509-13.
Altschul et al. (1997) Gapped BLAST and PSI-BLAST: a new generation of protein database search programs. Nucleic Acids Res 25:3389-3402.
Amidon et al. (1995) A theoretical basis for a biopharmaceutic drug classification: the correlation of in vitro drug product dissolution and in vivo bioavailability. Pharm Res 12:413-420.
Bird et al. (1988) Single-chain antigen-binding proteins. Science 242:423-426
Crielaard et al. (2012) Liposomes as carriers for colchicine-derived prodrugs: Vascular disrupting nanomedicines with tailorable drug release kinetics. European Journal of Pharmaceutical Sciences 45(4):429-435.
Deftereos et al. (2022) Colchicine in Cardiovascular Disease: In-Depth Review. Circulation 145(1):61-78.
Devereux et al. (1984) A comprehensive set of sequence analysis programs for the VAX. Nucl Acids Res 12:387.
Elsewedy et al. (2022) Enhancement of Anti-Inflammatory Activity of Optimized Niosomal Colchicine Loaded into Jojoba Oil-Based Emulgel Using Response Surface Methodology. Gels 8(1):16.
Gennaro (ed.) (1985) Remington's Pharmaceutical Sciences, Mack Publishing Co., Easton, Pennsylvania, United States of America.
Gennaro (ed.) (1990) Remington's Pharmaceutical Sciences, 18th ed., Mack Pub. Co., Easton, Pennsylvania, United States of America,
Gennaro (ed.) (2003) Remington: The Science and Practice of Pharmacy, 20th edition Lippincott, Williams & Wilkins, Philadelphia, Pennsylvania, United States of America.
Gross & Mienhofer (eds.) (1981) The Peptides, Vol. 3, Academic Press, New York, New York, United States of America. pages 3-88.
Harlow & Lane (1988) Antibodies, A Laboratory Manual, Cold Spring Harbor Publications, Cold Spring Harbor, New York, United States of America.
Huston et al. (1988) Protein engineering of antibody binding sites: recovery of specific activity in an anti-digoxin single-chain Fv analogue produced in Escherichia coli. Proc Natl Acad Sci USA 85:5879.
Jones et al (1986) Replacing the complementarity-determining regions in a human antibody with those from a mouse. Nature 321:522.
Joshi et al. (2017) Fabrication and in-vivo evaluation of lipid nanocarriers based transdermal patch of colchicine. Journal of Drug Delivery Science and Technology 41:444-453.
Karlin & Altschul (1990) Methods for assessing the statistical significance of molecular sequence features by using general scoring schemes. Proc Natl Acad Sci USA 87(6):2264-2268.
Karlin & Altschul (1993) Applications and statistics for multiple high-scoring segments in molecular sequences. Proc Natl Acad Sci USA 90(12):5873-5877.
Koseki et al. (2016) Drug release is determined by the chain length of fatty acid-conjugated anticancer agent as one component of nano-prodrug. Bulletin of the Chemical Society of Japan 89(5):540-545.
Mohamed et al. (2020) Colchicine mesoporous silica nanoparticles/hydrogel composite loaded cotton patches as a new encapsulator system for transdermal osteoarthritis management. International Journal of Biological Macromolecules 164:1149-1163.
Nasr et al. (2020) Formulation and evaluation of cubosomes containing colchicine for transdermal delivery. Drug Delivery and Translational Research 10(5):1302-1313.
Parashar et al. (2022) Appraisal of anti-gout potential of colchicine-loaded chitosan nanoparticle gel in uric acid-induced gout animal model. Archives of Physiology and Biochemistry 128(2):547-557.
Porter et al. (2007) Lipids and lipid-based formulations: Optimizing the oral delivery of lipophilic drugs. Nature Reviews Drug Discovery 6(3):231-248.
Riechmann et al. (1988) Reshaping human antibodies for therapy. Nature 332(6162):323-327.
Roubille et al. (2013) Colchicine: An Old Wine in a New Bottle? Anti-Inflammatory & Anti-Allergy Agents in Medicinal Chemistry 12(1):14-23.
Shchegravina et al. (2019) Phospholipidic Colchicinoids as Promising Prodrugs Incorporated into Enzyme-Responsive Liposomes: Chemical, Biophysical, and Enzymological Aspects. Bioconjugate Chemistry 30(4):1098-1113.
Singh et al. (2021) Role of Chain Length and Degree of Unsaturation of Fatty Acids in the Physicochemical and Pharmacological Behavior of Drug-Fatty Acid Conjugates in Diabetes. Journal of Medicinal Chemistry 64(19):14217-14229.
U.S. Patent Application Publication Nos. 2003/0017534, 2018/0298087, 2018/0312588, 2018/0346564, 2019/0151448.
U.S. Pat. Nos. 4,816,567; 5,482,856; 6,479,284; 6,677,436; 7,060,808; 7,906,625; 8,398,980; 8,436,150; 8,796,439; 10,253,111.
Wang et al. (2022) Colchicine-Containing Nanoparticles Attenuates Acute Myocardial Infarction Injury by Inhibiting Inflammation. Cardiovascular Drugs and Therapy 36(6):1075-1089.
Winter & Milstein (1991) Man-made antibodies. Nature 349(6307):293-299.
Zhang et al. (2020) A novel colchicine/gadolinium-loading tubulin self-assembly nanocarrier for MR imaging and chemotherapy of glioma. Nanotechnology 31:255601.

It will be understood that various details of the presently disclosed subject matter can be changed without departing from the scope of the presently disclosed subject matter. Furthermore, the foregoing description is for the purpose of illustration only, and not for the purpose of limitation.

1. A composition comprising colchicine, hydroxychloroquine, cytarbine, a statins, optionally rosuvastatin and/or simvastatin, and/or a prodrug thereof conjugated to a fatty-acyl group, wherein the fatty-acyl group is selected from the group consisting of saturated, monounsaturated, and polyunsaturated fatty acids of from 1-30 carbons, optionally wherein the fatty acyl group comprises a behenic acid group, an oleic acid group, a stearic acid group, or a palmitic acid group, and further optionally wherein the colchicine and/or a prodrug thereof conjugated to the fatty-acyl group is encapsulated by a nanoparticle (NP), optionally a liposome.

2. The composition of claim 1, wherein the colchicine and/or the prodrug thereof is conjugated to the fatty-acyl group at the acetamide position of a B-ring of the colchicine or the prodrug thereof.

3. The composition of claim 1, wherein the liposomes comprises a lipid component comprising DSPC, DOPE, and DSPE-PEG, and optionally further comprises about 5% cholesterol.

4. The composition of claim 1, wherein the colchicine and/or the prodrug thereof conjugated to the fatty-acyl group is present in the liposomes in an amount of at least about 400 μg/mL, 500 μg/mL, 600 μg/mL, or more than 600 μg/mL.

5. The composition of claim 1, wherein the fatty-acyl group is conjugated to the colchicine and/or the prodrug thereof via a linker.

6. The composition of claim 1, wherein nanoparticle (NP), optionally the liposome, further comprises a targeting agent, optionally wherein the targeting agent binds to E-selectin, L-selectin, or both, or comprises a D-α-tocopheryl polyethylene glycol succinate moiety to target the composition to a target of interest.

7. A method for preparing a fatty-acyl conjugated colchicine and/or colchicine prodrug, the method comprising:
(a) reacting colchicine and/or a colchicine prodrug with N-dimethyl amino pyridine (DMAP) dissolved in acetonitrile with di-tert-butyl decarbonate (Boc₂O) and triethyl amine at room temperature under argon for at least about 4 hours, optionally 6 hours, optionally wherein the cochicine and/or the colchicine prodrug and the DMAP are in equimolar amounts, and relative to the colchicine and/or the colchicine prodrug, the Boc₂O is present in 5-6 equivalents and the triethyl amine is present in 2 equivalents;
(b) adding excess Boc₂O and continuing the reaction for about an hour at 80° C. to produce a Boc-protected colchicine and/or colchicine prodrug, optionally wherein the excess Boc₂O added is in an amount of about 0.5-0.6 equivalents;
(c) reacting purified Boc-protected colchicine and/or colchicine prodrug dissolved in methanol with NaOMe in methanol at 4° C. for about 1 hour to produce N-Boc-deacetylcolchicine and/or a N-Boc-deacetylcolchicine prodrug;
(d) reacting the purified N-Boc-deacetylcolchicine and/or N-Boc-deacetylcolchicine prodrug dissolved in dichloromethane (DCM) with trifluoroacetic acid (TFA) for about 2 hours at room temperature to produce N-deacetylcolchicine and/or an N-deacetylcolchicine prodrug;
(e) reacting the N-deacetylcolchicine and/or the deacetylcolchicine prodrug with glycolic acid (optionally 1.0 equivalent), NHS, Et₃N, and DIC in DCM for about 24 hours at room temperature to produce colchifoline from the N-deacetylcolchicine and/or a colchifoline derivative from the deacetylcolchicine prodrug; and
(f) reacting the colchifoline and/or the deacetylcolchicine prodrug with a fatty-acyl group in EDC·HCl and DMAP in DCM at room temperature,
whereby a fatty-acyl conjugated colchicine and/or colchicine prodrug is prepared.

8. A method for preparing a liposomal nanoparticle-encapsulated colchicine and/or colchicine prodrug, the method comprising encapsulating a colchicine-fatty acyl conjugate and/or a colchicine prodrug-fatty acyl conjugate in a liposomal nanoparticle.

9. The method of claim 8, wherein the colchicine-fatty acyl conjugate and/or the colchicine prodrug-fatty acyl conjugate is a colchicine-behenic acid conjugate and/or a colchicine prodrug-behenic acid conjugate, a colchicine-oleic acid conjugate and/or a colchicine prodrug-oleic acid conjugate, and/or a colchicine-stearic acid conjugate and/or a colchicine prodrug-stearic acid conjugate.

10. The method of claim 8, wherein the nanoliposome comprises a lipid component comprising DSPC, DOPE, and DSPE-PEG, and optionally comprises about 5% cholesterol.

11. The method of claim 8, wherein the colchicine and/or the colchicine prodrug is present in the liposome in an amount of at least about 400 μg/mL, 500 μg/mL, 600 μg/mL, or more than 600 μg/mL.

12. A method for treating and/or preventing an inflammatory disease, disorder, and/or condition, a cardiovascular disease, disorder, and/or condition, and/or an infection with a virus and/or other microbe, the method comprising administering to a subject in need thereof an effective amount of a composition of claim 1.

13. The method of claim 12, wherein the inflammatory disease, disorder, or condition is selected from the group consisting of gout, familial Mediterranean fever, recurrent pericarditis, arthritis, asthma, dermatitis, psoriasis, cystic fibrosis, post transplantation late and chronic solid organ rejection, multiple sclerosis, systemic lupus erythematosus, inflammatory bowel diseases, autoimmune diabetes, diabetic retinopathy, diabetic nephropathy, diabetic vasculopathy, ocular inflammation, uveitis, rhinitis, ischemia-reperfusion injury, post-angioplasty restenosis, chronic obstructive pulmonary disease (COPD), glomerulonephritis, Graves disease, gastrointestinal allergies, conjunctivitis, atherosclerosis, coronary artery disease, angina, and small artery disease.

14. The method of claim 13, wherein the cardiovascular disease is selected from the group consisting of cholesterol- or lipid-related disorders, include, but are not limited to acute coronary syndrome, angina, arteriosclerosis, atherosclerosis, carotid atherosclerosis, cerebrovascular disease, cerebral infarction, congestive heart failure, congenital heart disease, coronary heart disease, coronary artery disease, coronary plaque stabilization, dyslipidemias, dyslipoproteinemias, endothelium dysfunctions, familial hypercholeasterolemia, familial combined hyperlipidemia, hypoalphalipoproteinemia, hypertriglyceridemia, hyperbetalipoproteinemia, hypercholesterolemia, hypertension, hyperlipidemia, intermittent claudication, ischemia, ischemia reperfusion injury, ischemic heart diseases, cardiac ischemia, metabolic syndrome, multi-infarct dementia, myocardial infarction, obesity, peripheral vascular disease, reperfusion injury, restenosis, renal artery atherosclerosis, rheumatic heart disease, stroke, thrombotic disorder, transitory ischemic attacks, and lipoprotein abnormalities associated with Alzheimer's disease, obesity, diabetes mellitus, syndrome X, impotence, multiple sclerosis, Parkinson's disease, and inflammatory diseases.

15. The method of claim 12, wherein the effective amount of the composition is administered to the subject intraperitoneally, orally, intravenously, intramuscularly, subcutaneously, or a combination thereof.

16. The method of claim 12, wherein the composition is targeted to a cell, tissue, or organ of interest by conjugating the nanoparticle to a moiety that binds to a target molecule present on or in the cell, tissue, or organ.

17. The method of claim 16, wherein the moeity is an antibody or a fragment thereof that binds to the target.

18. The method of claim 17, wherein:
(i) the moiety is an anti-L-selectin antibody that targets the composition to a leukocyte; and/or
(ii) the moiety is an anti-E-selectin moiety that targets the composition to an atherosclerotic lesion; and/or
(iii) moiety is an anti-E-selectin moiety that targets the composition to an endothelial cell,
whereby the inflammatory disease, disorder, and/or condition and/or the cardiovascular disease, disorder, and/or condition is treated or prevented.

19. Use of the composition of claim 1 for treating and/or preventing an inflammatory disease, disorder, and/or condition and/or a cardiovascular disease, disorder, and/or condition.

20. A composition for use in treating and/or preventing an inflammatory disease, disorder, and/or condition and/or a cardiovascular disease, disorder, and/or condition, the composition comprising colchicine and/or a prodrug thereof conjugated to a fatty-acyl group, wherein the fatty-acyl group is optionally a behenic acid group, an oleic acid group, and/or a stearic acid group, further optionally wherein the colchicine and/or a prodrug thereof conjugated to the fatty-acyl group is encapsulated by a nanoparticle (NP), optionally a liposome.

21. The composition for use of claim 20, wherein the colchicine and/or the prodrug thereof is conjugated to the fatty-acyl group at the acetamide position of a B-ring of the colchicine or the prodrug thereof.

22. The composition for use of claim 20, wherein the liposome comprises a lipid component comprising DSPC, DOPE, and DSPE-PEG, and optionally further comprises about 5% cholesterol.

23. The composition for use of claim 20, wherein the colchicine and/or the prodrug thereof conjugated to the fatty-acyl group is present in the liposome in an amount of at least about 400 μg/mL, 500 μg/mL, 600 μg/mL, or more than 600 μg/mL.

24. The composition for use of claim 20, wherein the fatty-acyl group is conjugated to the colchicine and/or the prodrug thereof via a linker.

25. A method for improving entrapment efficiency, retention time, and/or release profile of a hydrophilic drug, optionally colchicine in a liposome, optionally a nanoliposome, the method comprising conjugating a fatty acyl group to the hydrophilic drug and encapsulating the same in the liposome, wherein the entrapment efficiency, the retention time, and/or the release profile of the hydrophilic drug with respect to the liposome is improved relative to the entrapment efficiency, the retention time, and/or the release profile of the hydrophilic drug lacking the fatty acyl group with respect to the liposome.
